(19) United States
(12) Patent Application Publication
(10) Pub. No.: US 2023/0061798 A1
(43) Pub. Date: Mar. 2, 2023

(54) METHOD FOR CULTURING FACTOR-INTRODUCED CELLS

(71) Applicants: Koji TANABE, Palo Alto, CA (US); I Peace, Inc., Palo Alto, CA (US)

(72) Inventors: Koji TANABE, Palo Alto, CA (US); Kenta SUTO, Palo Alto, CA (US)

(73) Assignee: I Peace, Inc., Palo Alto, CA (US)

(21) Appl. No.: 17/759,327

(22) PCT Filed: Jan. 22, 2021
(86) PCT No.: PCT/JP2021/002336

§ 371 Date: Jul. 22, 2022

Related U.S. Application Data
(60) Provisional application No. 63/131,451, filed on Dec. 29, 2020.
(60) Provisional application No. 63/025,814, filed on May 15, 2020.
(60) Provisional application No. 62/988,131, filed on Mar. 11, 2020.
(60) Provisional application No. 62/965,669, filed on Jan. 24, 2020.

(51) Int. Cl. C12N 5/074

(52) U.S. Cl. CPC C12N 5/0696; C12N 2506/1307; C12N 2533/52; C12N 2501/603; C12N 2501/602; C12N 2501/604; C12N 2501/606

(57) ABSTRACT

According to the present disclosure, there is provided a method for culturing factor-introduced cells, the method including culturing factor-introduced cells and recovering the factor-introduced cells and seeding at least part of the recovered cells in a medium for seeding. In addition, there is provided a method for culturing factor-introduced cells, the method including culturing factor-introduced cells and inducing the factor-introduced cells to somatic cells different from pluripotent stem cells without passaging.

### TECHNICAL FIELD

The present invention relates to a cell technique and a method for culturing factor-introduced cells.

### BACKGROUND ART

Induced pluripotent stem (iPS) cells are cells having two characteristic abilities. One is an ability to transform into any cells that constitute the body. The other is to have a semi-permanent proliferative ability. Since iPS cells have these two abilities, these cells can be applied to a transplantation treatment without being rejected by producing iPS cells from their own somatic cells and transforming them into target somatic cells. Therefore, iPS cells are considered to be a key technology for regenerative medicine (for example, refer to Patent Documents 1 to 4 and Non-Patent Documents 1 and 2). In the prior art, when a reprogramming factor is introduced into cells to induce the cells to iPS cells, stem-cell-like colonies are picked up with a pipette and passaged while being observed under a microscope or the like.

### CITATION LIST

### Patent Document

Patent Document 1: WO 2000/70070
Patent Document 2: WO 2010/008054
Patent Document 3: WO 2012/029770
Patent Document 4: WO 2015/046229

### Non-Patent Document

Non-Patent Document 1: Nature 448, 313-317
Non-Patent Document 2: Nature Biotechnol 26(3): 313-315, 2008.

### SUMMARY

### Technical Problem

There is a demand for a method for efficiently culturing factor-introduced cells. One object of the present invention is to provide a method for efficiently culturing factor-introduced cells.

### Solution to Problem

According to an aspect of the present invention, there is provided a method for culturing factor-introduced cells without cloning cells.

According to an aspect of the present invention, there is provided a method for culturing factor-introduced cells, the method including seeding factor-introduced cells without cloning.

According to an aspect of the present invention, there is provided a method for culturing factor-introduced cells, the method including separating factor-introduced cells from an incubator and mixing and seeding at least part of the separated cells. The separated cells may be mixed.

According to an aspect of the present invention, there is provided a method for culturing factor-introduced cells, the method including recovering factor-introduced cells from an incubator and mixing and seeding at least part of the recovered cells. The recovered cells may be mixed.

According to an aspect of the present invention, there is provided a method for culturing factor-introduced cells without picking up each of a plurality of colonies formed by the factor-introduced cells.

According to an aspect of the present invention, there is provided a method for culturing factor-introduced cells, the method including mixing and seeding cells which are derived from different single cells, and which are factor-introduced cells.

In the method, in the seeding, the cells may not be cloned.

In the method, in the seeding, the factor-introduced cells may be mixed.

In the method, in the seeding, clones of the factor-introduced cells may be mixed.

In the method, in the seeding, different clones of the factor-introduced cells may be mixed.

The method may not include isolating a plurality of colonies, which are formed by the factor-introduced cells, before the seeding.

In the method, before the seeding, it is not necessary to pick up each of a plurality of colonies formed by the factor-introduced cells.

In the method, in the seeding, a plurality of colonies formed by the factor-introduced cells may be mixed with each other.

The method may not include cloning a single colony, which is formed by the factor-introduced cells, before the seeding.

The method may not include picking up colonies formed by the factor-introduced cells.

In the method, cells which are attached to an incubator and which are the factor-introduced cells may be recovered and at least part of the recovered cells may be seeded and passaged in a medium.

In the method, the factor-introduced cells may be seeded without distinguishing the cells according to a gene expression state thereof.

In the method, the factor-introduced cells may be seeded without distinguishing the cells according to a degree of reprogramming.

The method may further include expansion-culturing the factor-introduced cells in two-dimensional culture.

The method may further include expansion-culturing the factor-introduced cells in three-dimensional culture.

The method may further include deriving stem cells from the factor-introduced cells.

In the method, the seeded cells may be induced to pluripotent stem cells.

The method may further include inducing pluripotent stem cells to somatic cells.

The method may further include freezing the factor-introduced cells after seeding.

The method may further include differentiating the factor-introduced cells into at least one selected from among the endoderm, the mesoderm, and the ectoderm after seeding.

The method may further include forming at least one selected from among embryoid bodies, organoids, and spheres from the factor-introduced cells after seeding.

The method may further include inducing the factor-introduced cells to somatic cells different from pluripotent stem cells after seeding.

The method may further include cloning cells induced to somatic cells after a process of inducement to somatic cells.

The method may further include performing a gene editing process on the factor-introduced cells.

In the method, the factor-introduced cells may be derived from blood cells or fibroblasts.

In the method, the cells to which the factor is introduced may be cells contained in urine.

In the method, the cells to which the factor is introduced may be bladder epithelial cells.

The method may further include collecting cells into which the factor is introduced from urine.

In the method, the factor-introduced cells may be derived from a plurality of humans or a plurality of non-human animals.

In the method, the factor-introduced cells may be cultured in a closed incubator.

In the method, during passage, the cells may be seeded at a low concentration.

In the method, the low concentration may be 0.25×10⁴ cells/cm² or less.

In the method, the low concentration may be a concentration at which 11 or more of the seeded cells do not come into contact with each other.

In the method, the low concentration may be 5% or less confluency.

In the method, the factor may be RNA.

In the method, the factor may be introduced into the cells by a lipofection method.

In the method, the factor may be introduced into the cells using a viral vector.

In the method, the viral vector may be an RNA viral vector.

In the method, the RNA viral vector may be a Sendai viral vector.

In addition, according to an aspect of the present invention, there is provided a method for culturing factor-introduced cells including culturing factor-introduced cells and inducing the factor-introduced cells to somatic cells different from pluripotent stem cells without passaging.

The method may further include expansion-culturing the factor-introduced cells in two-dimensional culture.

The method may further include expansion-culturing the factor-introduced cells in three-dimensional culture.

The method may further include freezing the factor-introduced cells.

The method may further include differentiating the factor-introduced cells into at least one selected from among the endoderm, the mesoderm, and the ectoderm.

The method may further include performing a gene editing process on the factor-introduced cells.

In the method, the factor-introduced cells may be derived from blood cells or fibroblasts.

In the method, the factor-introduced cells may be derived from a plurality of humans or a plurality of non-human animals.

In the method, the factor-introduced cells may be cultured in a closed incubator.

In the method, the factor may be RNA.

In the method, the factor may be introduced into the cells by a lipofection method.

In the method, the factor may be introduced into the cells using a viral vector.

In the method, the viral vector may be an RNA viral vector.

In the method, the RNA viral vector may be a Sendai viral vector.

### Advantageous Effects of Invention

According to the present invention, it is possible to provide a method for efficiently culturing factor-introduced cells.

### BRIEF DESCRIPTION OF DRAWINGS

FIG. 1 shows graphs of the measurement results obtained by a flow cytometer according to Example 1.

FIG. 2 is a graph showing PCR results according to Example 1.

FIG. 3 shows images of TRA1-60 positive cells according to Example 1.

FIG. 4 shows graphs of clonal efficiency according to Example 1 and Comparative Example 1.

FIG. 5 is a graph showing the number of cells according to Example 2 and Comparative Example 2.

FIG. 6 is a graph showing the number of colonies according to Example 2 and Comparative Example 2.

FIG. 7 shows graphs of the number of clamps and the number of cells according to Example 3 and Comparative Example 3.

FIG. 8 is a table showing whether myocardial pulsation occurs according to Example 4 and Comparative Example 4.

FIG. 9 is a graph showing the positive rate of cTnT according to Example 4 and Comparative Example 4.

FIG. 10 shows graphs of the number of cells and the positive rate of PSA-NCAM according to Example 5 and Comparative Example 5.

FIG. 11 shows graphs of the positive rate of SOX1 and the positive rate of OTX2 according to Example 6 and Comparative Example 6.

FIG. 12 shows graphs of the positive rate of HAND1 and the positive rate of SOX17 according to Example 6 and Comparative Example 6.

FIG. 13 shows graphs of the measurement results obtained by a flow cytometer according to Reference Example 1.

FIG. 14 is an image showing TRA1-60 positive cells according to Reference Example 1.

FIG. 15 shows graphs of the measurement results obtained by a flow cytometer according to Reference Example 2.

FIG. 16 shows graphs of the measurement results obtained by a flow cytometer according to Reference Example 3.

FIG. 17 shows graphs of the measurement results obtained by a flow cytometer according to Reference Example 4.

FIG. 18 is an image showing cells 15 days after infection according to Reference Example 4.

FIG. 19 shows graphs of the measurement results obtained by a flow cytometer according to Reference Example 4.

FIG. 20 is an image showing cells in the first passage according to Reference Example 4.

FIG. 21 shows graphs of the measurement results obtained by a flow cytometer according to Reference Example 5.

FIG. 22 is an image showing TRA1-60 positive cells according to Reference Example 5.

FIG. 23 shows graphs of the measurement results obtained by a flow cytometer according to Reference Example 6.

FIG. 24 is a graph showing PCR results according to Reference Example 6.

FIG. 25 is an image showing TRA1-60 positive cells according to Reference Example 6.

FIG. 26 is a graph showing PCR results according to Example 7.

FIG. 27 shows images of TRA1-60 positive cells according to Example 7.

FIG. 28 is an image of nervous system cells according to Example 8.

FIG. 29 shows images of teratomas according to Example 9.

FIG. 30 is an image of iPS-cell-like colonies according to Example 10.

FIG. 31 shows images of Oct3/4 positive cells and Nanog positive cells according to Example 10.

FIG. 32 is a dot plot obtained by a flow cytometer according to Example 10.

FIG. 33 is an image of cardiomyocytes according to Example 10.

FIG. 34 shows images of Munch13 positive cells and vGlut positive cells according to Example 10.

FIG. 35 is an image of urine-derived cells according to Example 11.

FIG. 36 is an image of urine-derived cells according to Example 11.

FIG. 37 shows images of urine-derived cells transfected with RNA encoding GFP according to Example 12.

FIG. 38 shows images of urine-derived cells into which the reprogramming factor is introduced according to Example 13.

FIG. 39 is an image of urine-derived cells into which the reprogramming factor is introduced according to Example 14.

FIG. 40 shows dot plots obtained by a flow cytometer according to Example 15.

FIG. 41 shows images of urine-derived cells into which the reprogramming factor is introduced according to Example 16.

FIG. 42 shows images of urine-derived cells into which the reprogramming factor is introduced according to Example 16.

### DESCRIPTION OF EMBODIMENTS

Embodiments of the present invention will be described below in detail. Here, the following embodiments exemplify a device and a method for embodying the technical ideas of the invention, and the technical ideas of the invention do not specify the combination of constituent members or the like as in the following. The technical ideas of the invention can be variously modified within the scope of the claims.

A method for culturing reprogramming factor-introduced cells according to an embodiment includes culturing cells into which the reprogramming factor is introduced and passaging without cloning the cells into which the reprogramming factor is introduced. In addition, the method for culturing reprogramming factor-introduced cells according to the embodiment includes culturing cells into which the reprogramming factor is introduced, and separating the cells into which the reprogramming factor is introduced from an incubator and mixing and passaging at least some of the separated cells. In addition, the method for culturing reprogramming factor-introduced cells according to the embodiment includes culturing cells into which the reprogramming factor is introduced, recovering the cells into which the reprogramming factor is introduced mixing at least some of the recovered cells, and seeding them in a medium for passaging. In addition, the method for culturing reprogramming factor-introduced cells according to the embodiment includes mixing and seeding cells derived from different single cells, which are cells into which the reprogramming factor is introduced. The cells into which the reprogramming factor is introduced are seeded and induced to, for example, pluripotent stem cells. The pluripotent stem cells are, for example, iPS cells.

Cells into which the reprogramming factor is introduced are not particularly limited, and examples thereof include fibroblasts, blood cells, dental pulp stem cells, keratinocytes, dermal papilla cells, oral epithelial cells, and somatic prestem cells. Cells into which the reprogramming factor is introduced may be cells contained in urine. Examples of cells contained in urine include bladder epithelial cells. Cells into which the reprogramming factor is introduced may be cells derived from humans or derived from non-human animals. Cells into which the reprogramming factor is introduced may be derived from one human or derived from a plurality of humans. Cells into which the reprogramming factor is introduced may be derived from one non-human animals or derived from a plurality of non-human animals.

Blood cells are isolated from blood. The blood is, for example, peripheral blood or cord blood, but is not limited thereto. Blood may be collected from an adult or a minor. During blood sampling, an anticoagulant such as ethylene-diamine-tetraacetic acid (EDTA), heparin, and a biological preparation standard blood preservative solution A (ACD-A) is used.

Blood cells are nucleated cells, for example, mononuclear cells (monocytes), neutrophilic leukocytes, macrophages, eosinophilic leukocytes, basophil leukocytes, and lymphocytes, and do not include red blood cells, granulocytes, and platelets. Blood cells may be, for example, endothelial progenitor cells, blood stem/progenitor cells, T cells, or B cells. T cells are, for example, αβT cells.

Mononuclear cells are isolated from blood using a medium for isolating blood cells, a centrifugal device or the like. A method for isolating mononuclear cells when Ficoll (GE Healthcare) is used as a medium for isolating blood cells is as follows.

Since the isolation accuracy of mononuclear cells tends to deteriorate at a low temperature, the centrifuge is set at 4° C. to 42° C., preferably 18° C. 10 μL to 50 mL of blood is sampled from an adult or minor human, a chelating agent containing EDTA is added to the blood to prevent the blood from coagulating, and is mixed gently. In addition, 5 mL of a medium for isolating human lymphocytes (Ficoll-Paque PREMIUM, GE Healthcare Japan) is dispensed into two 15 mL tubes. 5 mL of PBS is added to 5 mL of blood for dilution, and 5 mL layers are placed on the medium for isolating human lymphocytes in each of the tubes. In this case, the diluted blood is slowly added onto the medium along the tube wall of the tube to prevent disturbance of the interface.

The solution in the tube is centrifuged at 10×g to 1,000×g, and preferably, 400×g, at 4° C. to 42° C., preferably 18° C., for 5 minutes to 2 hours, preferably for 30 minutes. After centrifugation, a cloudy white intermediate layer appears in the tube. The cloudy white intermediate layer contains mononuclear cells. The cloudy white intermediate layer in the tube is slowly recovered with a Pipeteman and is transferred to a new 15 mL tube. In this case, the lower layer should not be sucked up. About 1 mL of the cloudy white intermediate layer can be recovered from one tube. Two intermediate layers are transferred together into one tube.

1 mL to 48 mL, preferably 12 mL of PBS is added to the recovered mononuclear cells, and the solution is additionally centrifuged at 10×g to 1,000×g, preferably 200×g, at 4° C. to 42° C., preferably 18° C., for 1 minute to 60 minutes, preferably 10 minutes. Then, the supernatant of the solution is sucked up and removed using an aspirator, and 1 mL to 12 mL, preferably 3 mL of a serum-free hematopoietic cell medium of known composition (X-VIVO (registered trademark) 10, Lonza) is added for suspension therein to obtain a mononuclear cell suspension. Of this, 10 μL of a mononuclear cell suspension is stained with trypan blue and counting is performed on a hemacytometer.

A method for isolating mononuclear cells when a Vacutainer (registered trademark, BD) is used as a blood collection tube is as follows.

Since the isolation accuracy of mononuclear cells tends to deteriorate at a low temperature, the centrifuge is set to 4° C. to 42° C., preferably 18° C. 8 mL of blood is sampled from an adult or minor human using a blood collection tube (Vacutainer (registered trademark), BD), mixed by inversion and mixed with an anticoagulant. Then, the balance is adjusted, and the solution is centrifuged at 4° C. to 42° C., preferably 18° C., at 100×g to 3,000×g, preferably 1,500×g to 1,800×g with a swing rotor for 1 minute to 60 minutes, preferably 20 minutes. After centrifugation, the upper layer, which is a plasma layer, is removed and pipetting is performed to suspend the mononuclear cell layer and blood cells adhered to the gel to obtain a suspension. The obtained suspension is transferred to another 15 mL tube.

1 mL to 14 mL, preferably 12 mL of PBS is added to the suspension in a 15 mL tube, and the suspension is centrifuged at 4° C. to 42° C., preferably 18° C., at 100×g to 3,000×g, preferably 200×g for 1 minute to 60 minutes, preferably 5 minutes. After centrifugation, the supernatant is removed with an aspirator. In addition, a hemolytic agent (PharmLyse (registered trademark), 10-fold concentration, BD) is diluted to a 1-fold concentration with sterilized water. The pellet in the 15 mL tube is loosened by tapping, and 1 mL to 14 mL, preferably 1 mL of a hemolytic agent is added. Then, light is blocked therefrom and the solution is left for 1 minute to 60 minutes, preferably 1 minute at room temperature.

Next, 1 mL to 14 mL, preferably 12 mL of PBS is added to a 15 mL tube, and centrifugation is performed at 4° C. to 42° C., preferably room temperature, at 100×g to 3,000×g, preferably 200×g for 1 minute to 60 minutes, or 5 minutes. After centrifugation, the supernatant is removed with an aspirator, and 1 mL to 15 mL, and preferably 3 mL of a serum-free hematopoietic cell medium of known composition (X-VIVO (registered trademark) 10, Lonza) is added for suspension therein to obtain a mononuclear cell suspension. Of this, 10 μL of a mononuclear cell suspension is stained with trypan blue and counting is performed on a hemacytometer.

The method for isolating mononuclear cells from blood is not limited to the above method, and for example, mononuclear cells may be isolated from blood using a dialysis membrane. In addition, Purecell Select System for whole blood mononuclear cell concentration (registered trademark, PALL), a purifier for removing blood cell cells (Cellsorba E, registered trademark, Asahi Kasei), and a filter such as a white blood cell removal filter for platelet preparation (Sepacell PL, registered trademark, PLX-5B-SCD, Asahi Kasei) can also be used.

Mononuclear cells may be isolated using a red blood cell isolating agent that can isolate nucleated cells by gravitational precipitation or centrifugation of red blood cells. Examples of red blood cell isolating agents include HetaSep (registered trademark, STEMCELL Technologies) and HES40 (NIPRO).

In addition, CTL-UP1 (commercially available from Cellular Technology Limited), PBMC-001 (commercially available from Sanguine Biosciences), or the like may be used as mononuclear cells.

Alternatively, regarding the blood cells, blood cells that are cryopreserved using a cell cryopreservation solution such as Cellbanker 1, Stem-Cellbanker GMP grade, or Stem-Cellbanker DMSO free GMP grade (ZENOAQ) may be thawed and used.

When thawing mononuclear cells, first, 1 mL to 15 mL, preferably 8 mL of a serum-free hematopoietic cell medium of known composition (X-VIVO (registered trademark) 10, Lonza) is put into a 15 mL tube, the tube containing frozen mononuclear cells is placed in a warm bath at 4° C. to 42° C., preferably 37° C., and the mononuclear cells start to melt. Then, with the remaining ice, the tube containing mononuclear cells is pulled out of the warm bath, and the mononuclear cells are transferred to a tube containing a serum-free hematopoietic cell medium of known composition. Of this, 10 μL of a mononuclear cell suspension is stained with trypan blue and counting is performed on a hemacytometer.

Blood cells may be isolated based on a cell surface marker. Blood stem/progenitor cells are positive for CD34. T cells are positive for any of CD3, CD4, and CD8. B cells are positive for any of CD10, CD19, and CD20. Macrophages are positive for any of CD11b, CD68, and CD163. Blood stem/progenitor cells, T cells, or B cells are isolated from blood cells using, for example, an automatic magnetic cell isolating device and immunomagnetic beads. Alternatively, mononuclear cells isolated in advance may be prepared. However, blood cells that are not isolated based on a cell surface marker may be used.

CD34 positive cells are stem/progenitor cells, and tend to be easily reprogrammed. In addition, when iPS cells are prepared using T cells which are CD3 positive cells, since the iPS cells derived from T cells maintain a TCR recombination type, differentiation into T cells tends to be efficiently induced.

A method for isolating CD34 positive cells is as follows.

10 μL of IL-6 (100 μg/mL), 10 μL of SCF (300 μg/mL), 10 μL of TPO (300 μg/mL), 10 μL of FLT3 ligands (300 μg/mL), and 10 μL of IL-3 (10 μg/mL) are added to 10 mL of a serum-free medium (StemSpan H3000, STEMCELL Technologies) to prepare a blood cell medium (blood stem/progenitor cell medium).

1 mL to 6 mL, preferably 2 mL of a blood cell medium is put into one well of a 6-well plate. In addition, in order to prevent evaporation of the medium, 1 mL to 6 mL, and 2 mL of PBS are put into the other five wells. Then, the 6-well plate is placed in an incubator at 4° C. to 42° C., preferably 37° C. for warming.

10 μL to 1 mL, preferably 80 μL of EDTA (500 mmol/L) and 10 μL to 1 mL, preferably 200 μL of FBS are added to 20 mL of PBS to prepare a column buffer. A mononuclear cell suspension containing 1×10⁴ to 1×10⁹, preferably 2×10⁷ mononuclear cells is dispensed in a 15 mL tube, and the mononuclear cell suspension is centrifuged at 4° C. to 42° C., preferably 4° C., at 100×g to 3,000×g, preferably 300×g for 10 minutes. After centrifugation, the supernatant is removed, and mononuclear cells are suspended in 100 μL to 1 mL, preferably 300 μL of the column buffer.

10 μL to 1 mL, preferably 100 μL of an FcR blocking reagent (Miltenyi Biotec) and 10 μL to 1 mL, preferably 100 μL of a CD34 microbeads kit (Miltenyi Biotec) are added to the mononuclear cell suspension in the 15 mL tube. The FcR blocking reagent is used to increase the specificity of the microbead labeling. Then, the mononuclear cell suspension is mixed and left at 4° C. to 42° C., preferably 4° C. for 1 minute to 2 hours, preferably 30 minutes.

Next, 1 mL to 15 mL, preferably 10 mL of the column buffer is added to the mononuclear cell suspension in the 15 mL tube and diluted, and centrifugation is performed at 4° C. to 42° C., preferably 4° C., at 100×g to 1,000-g, preferably 300-g for 1 minute to 2 hours, preferably 10 minutes. After centrifugation, the supernatant in the 15 mL tube is removed with an aspirator, and 10 μL to 10 mL, preferably 500 μL of the column buffer is added for resuspension therein.

A column for an automatic magnetic cell isolating device (MS column, Miltenyi Biotec) is attached to an automatic magnetic cell isolating device (MiniMACS Separation Unit, Miltenyi Biotec), and 10 μL to 10 mL, preferably 500 μL of the column buffer is put into the column for washing. Next, mononuclear cells are put into the column. In addition, 10 μL to 10 mL, preferably 500 μL of the column buffer is put into the column, and the column is washed 1 to 10 times, preferably 3 times. Then, the column is removed from the automatic magnetic cell isolating device and put into a 15 mL tube. Next, 10 μL to 10 mL, preferably 1,000 μL of the column buffer is put into the column, and the syringe is immediately pushed to discharge CD34 positive cells to the 15 mL tube.

10 μL of a CD34 positive cell suspension is stained with trypan blue and the number of cells is counted on a blood cell counting chamber. In addition, the CD34 positive cell suspension in the 15 mL tube is centrifuged at 4° C. to 42° C., preferably 4° C., at 100×g to 1,000×g, preferably 300×g for 1 minute to 2 hours, preferably 10 minutes. After centrifugation, the supernatant is removed with an aspirator. In addition, CD34 positive cells are re-suspended in the warmed blood cell medium and the CD34 positive cells are sprinkled on a culture plate. Then, the CD34 positive cells are cultured for 6 days at 4° C. to 42° C., preferably 37° C., in 1% to 20%, preferably 5% CO₂. During this time, the medium does not have to be replaced.

A method for isolating cells with a marker other than CD34 is the same as a method for isolating CD34 positive cells.

The reprogramming factor introduced into cells is, for example, RNA. The RNA is, for example, mRNA. Examples of reprogramming factors introduced into cells include OCT RNA such as OCT3/4, SOX RNA such as SOX2, KLF RNA such as KLF4, and MYC RNA such as c-MYC. As reprogramming factor RNA, M₃O which is improved OCT3/4 may be used. In addition, the reprogramming factor RNA may further include RNA of at least one factor selected from the group consisting of LIN28A, FOXH1, LIN28B, GLIS1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF206, FOXD3, REX1, UTF1, KLF2, KLF5, ESRRB, miR-291-3p, miR-294, miR-295, NR5A1, NR5A2, TBX3, MBD3sh, TH2A, TH2B, and P53DD. These RNAs are available from TriLink. Here, although the gene symbols are denoted here as those of humans, this is not intended to limit the species by uppercase or lowercase letters. For example, denoting in all uppercase letters does not exclude inclusion of mouse or rat genes. However, in the examples, the gene symbols are shown according to the species actually used.

p53 is a cancer-suppression protein. The dominant negative mutant of p53 is not particularly limited as long as it can act competitively with a wild type p53 protein in somatic cells and inhibit a function of the wild type p53 protein. Examples of dominant negative mutants of p53 include p53P275S in which proline at position 275 (at position 278 in the case of humans) located in a DNA binding region of mouse p53 is point-mutated to serine, p53DD in which an amino acid at position 14-301 of mouse p53 (corresponding to position 11-304 in human p53) is deficient, p53S58A in which serine at position 58 of mouse p53 (at position 61 in the case of humans) is point-mutated to alanine, p53C135Y in which cysteine at position 135 of human p53 (at position 132 in the case of mice) is point-mutated to tyrosine, p53A135V in which alanine at position 135 of mouse p53 (at position 138 in the case of humans) is point-mutated to valine, p53R172H in which arginine at position 172 of mouse p53 (at position 175 in the case of humans) is point-mutated to histidine, p53R270H in which arginine at position 270 of mouse p53 (at position 273 in the case of humans) is point-mutated to histidine, and p53D278N in which aspartic acid at position 278 of mouse p53 (at position 281 in the case of humans) is point-mutated to asparagine.

RNA may be modified with pseudouridine (ψ) or 5-methyluridine (5meU). RNA may be polyadenylated.

RNA introduced into cells is, for example, single-stranded RNA, from which double-stranded RNA may be substantially removed. In addition, RNA introduced into cells is preferably substantially free of impurities such as small RNA and contaminants. The single-stranded RNA introduced into cells may be purified and/or concentrated in order to substantially remove double-stranded RNA. Examples of a method for purifying single-stranded RNA introduced into cells include a purification method using high performance liquid chromatography (HPLC). For example, 70% or more, 75% or more, 80% or more, 85% or more, or 90% or more of double-stranded RNA is removed through HPLC. Alternatively, in order to substantially remove double-stranded RNA, RNA introduced into cells may be treated with ribonuclease that decomposes double-stranded RNA.

RNA introduced into cells may further include RNA in the transactivation domain (TAD) of MYOD that is directly connected to the full length of OCT3/4 RNA.

The reprogramming factor is introduced into cells, for example, by a lipofection method. The lipofection method is a method in which a complex of a nucleic acid, which is a negatively charged substance, and a positively charged lipid, is formed by an electrical interaction, and the complex is incorporated into cells by endocytosis or membrane fusion. The lipofection method has advantages such as less damage to cells, excellent introduction efficiency, ease of operation, and less time-consumption.

For example, the reprogramming factor is introduced into cells cultured using an RNA transfection reagent. For example, when cells are mononuclear cells, immediately after mononuclear cells are isolated from blood, RNA may be introduced into the mononuclear cells.

Lipofectamine MessengerMAX (registered trademark, Thermo Fisher SCIENTIFIC) can be used as the RNA transfection reagent. Alternatively, regarding the RNA transfection reagent, for example, a lipofection reagent such as Lipofectamine (registered trademark) RNAiMAX (Thermo Fisher SCIENTIFIC), Lipofectamine StemTransfection Reagent (Thermo Fisher SCIENTIFIC), TransIT (Mirus), mRNA-In (MTI-GlobalStem), Stemfect RNA Transfection Kit (ReproCELL), Jet Messenger (Polyplus), Lipofectamin (registered trademark) 2000, Lipofectamin (registered trademark) 3000, NeonTransfection System (Thermo Fisher SCIENTIFIC), Stemfect RNA transfection reagent (Stemfect), NextFect (registered trademark) RNA Transfection Reagent (BiooSientific), Amaxa (registered product) Human T cell Nucleofector (registered product) kit (Lonza, VAPA-1002), Amaxa (registered product) Human CD34 cell Nucleofector (registered product) kit (Lonza, VAPA-1003), and ReproRNA (registered trademark) transfection reagent (STEMCELL Technologies) may be used.

Alternatively, for example, a reprogramming factor is introduced into cells using a viral vector. The viral vector may be an RNA viral vector. The RNA viral vector may be a Sendai viral vector. The Sendai viral vector may be a temperature-sensitive Sendai viral vector in which the stability of a viral nucleic acid decreases at a predetermined temperature or higher. The viral nucleic acid of the temperature-sensitive Sendai viral vector is stable below a predetermined temperature. The viral nucleic acid may be viral DNA or viral RNA. The viral nucleic acid may be a virus genome. The decrease in the stability of the viral nucleic acid may be at least one of decomposition of the viral nucleic acid and minimization of replication or proliferation of the viral nucleic acid. When the stability of the viral nucleic acid decreases, at least one of proliferation of the viral nucleic acid, the replication rate of the viral nucleic acid and the gene expression level decreases. The predetermined temperature is, for example, 36.5° C. or higher and 37.5° C. or lower, 36.6° C. or higher and 37.4° C. or lower, 36.7° C. or higher and 37.3° C. or lower, 36.8° C. or higher and 37.2° C. or lower, 36.9° C. or higher and 37.1° C. or lower, or 37° C. The stability of the viral nucleic acid of the temperature-sensitive Sendai viral vector, that is, at least one of the proliferation, the replication rate and the gene expression level, is high at a temperature lower than a predetermined temperature, and low at a predetermined temperature or higher. For example, in the temperature-sensitive Sendai viral vector, the proliferation rate or the gene expression level in cells cultured at 37° C. is ½ or less, ⅓ or less, ⅕ or less, 1/10 or less, or 1/20 or less with respect to the proliferation rate or the gene expression level in cells cultured at 32° C.

The Sendai virus encodes the N gene, P gene, M gene, F/HN gene, and L gene. The HN protein recognizes sialic acid on the cell surface when the Sendai virus attaches to cells and fixes virus particles to the cells. The F protein is cleaved and activated with extracellular proteases, and catalyzes the fusion of the fixed Sendai virus envelope and the cell membrane of target cells to establish infection. Along with its modified protein, that is, the P protein, the L protein catalyzes replication of viral nucleic acids in the cytoplasm after infection and transcription from the replicated multi-copy nucleic acids.

When the F gene is deleted in the Sendai viral vector, it is possible to restrict production of infectious virus particles from transgenic cells. In addition, when a mutation is introduced into at least one of the L gene and P gene, it is possible to make the Sendai viral vector temperature sensitive.

Examples of temperature-sensitive (TS) mutation of the Sendai virus include TS7 (Y942H/L1361C/L1558I mutation of the L protein), TS12 (D433A/R434A/K437A mutation of the P protein), TS13 (D433A/R434A/K437A mutation of the P protein and L1558I mutation of the L protein), TS14 (D433A/R434A/K437A mutation of the P protein and L1361C mutation of the L protein), and TS15 (D433A/R434A/K437A mutation of the P protein and L1361C/L1558I mutation of the L protein).

The Sendai viral vector is, for example, an F gene-deficient (ΔF) Sendai viral vector having G69E, T116A, and A183S mutations in the M protein, A262T, G264R, and K461G mutations in the HN protein, L511F mutation in the P protein, and N1197S and K1795E mutations in the L protein, which is a Sendai viral vector into which the TS7, TS12, TS13, TS14, or TS15 mutation is introduced. However, the temperature-sensitive mutation of the Sendai viral vector is not limited thereto.

The Sendai viral vector is, for example, SeV(PM)/TSΔF, SeV18+/TSΔF, or SeV(HNL)/TSΔF, and is a Sendai viral vector into which the TS7, TS12, TS13, TS14, or TS15 mutation is introduced. However, the temperature-sensitive mutation of the Sendai viral vector is not limited thereto.

The Sendai viral vector introduced into cells may be a combination of a temperature-sensitive Sendai viral vector and a temperature-insensitive Sendai viral vector. Alternatively, the Sendai viral vector introduced into cells may be a temperature-sensitive Sendai viral vector only and may not include a temperature-insensitive Sendai viral vector. For example, the Sendai viral vector introduced into cells may be only a temperature-sensitive Sendai viral vector into which the TS7, TS12, TS13, TS14, or TS15 mutation is introduced and may not include a temperature-insensitive Sendai viral vector. For example, the Sendai viral vector introduced into cells may be only a Sendai viral vector having a temperature sensitivity equal to or higher than that of a temperature-sensitive Sendai viral vector into which the TS7, TS12, TS13, TS14, or TS15 mutation is introduced, and may not include a temperature-insensitive Sendai viral vector. For example, the Sendai viral vector introduced into cells may be only a Sendai viral vector having a temperature sensitivity equal to or higher than that of a temperature-sensitive Sendai viral vector into which the TS7, TS12, TS13, TS14, or TS15 mutation is introduced, and may not include a Sendai viral vector having a lower temperature sensitivity than a temperature-sensitive Sendai viral vector into which the TS7, TS12, TS13, TS14, or TS15 mutation is introduced.

The Sendai viral vector introduced into cells carries arbitrary reprogramming factors. The Sendai viral vector introduced into cells may be, for example, a combination of a temperature-sensitive Sendai viral vector including KLF RNA, OCT RNA, and SOX RNA in that order and not including MYC RNA, and a temperature-sensitive Sendai viral vector including MYC RNA and not including KLF RNA, OCT RNA, and SOX RNA. However, the number, combination, and order of reprogramming factors carried on the Sendai viral vector are arbitrary, and are not particularly limited.

The Sendai viral vector introduced into cells may include a Sendai viral vector including KLF RNA and not including OCT RNA and SOX RNA. The Sendai viral vector including KLF RNA and not including OCT RNA and SOX RNA may be a temperature-sensitive Sendai viral vector or a temperature-insensitive Sendai viral vector. However, according to the findings of the inventors, if a temperature-insensitive Sendai viral vector is not introduced, the Sendai viral vector disappears earlier from the cells into which the Sendai viral vector is introduced.

The temperature-sensitive Sendai viral vector including KLF RNA, OCT RNA, and SOX RNA is, for example, an F gene-deficient Sendai viral vector having G69E, T116A, and A183S mutations in the M protein, A262T, G264R, and K461G mutations in the HN protein, L511F mutation in the P protein, and N1197S and K1795E mutations in the L protein, which is a Sendai viral vector including the TS7, TS12, TS13, TS14, or TS15 mutation. The temperature-sensitive mutation is, for example, TS7 or TS12, or TS12.

The temperature-sensitive Sendai viral vector including KLF RNA, OCT RNA, and SOX RNA is, for example, SeV(PM)KOS/TS7ΔF or SeV(PM)KOS/TS12ΔF, or SeV(PM)KOS/TS12ΔF.

The temperature-sensitive Sendai viral vector including MYC RNA is, for example, an F gene-deficient Sendai virus vector having G69E, T116A, and A183S mutations in the M protein, A262T, G264R, and K461G mutations in the HN protein, L511F mutation in the P protein, and N1197S and K1795E mutations in the L protein, which is a Sendai viral vector including the TS7, TS12, TS13, TS14, or TS15 mutation. The temperature-sensitive mutation is, for example, TS15.

The temperature-sensitive Sendai viral vector including MYC RNA is, for example, SeV(HNL)MYC/TS12ΔF, SeV(HNL)MYC/TS13ΔF, or SeV(HNL)MYC/TS15ΔF, or SeV(HNL)MYC/TS15ΔF.

The Sendai viral vector including KLF RNA and not including OCT RNA and SOX RNA is, for example, an F gene-deficient Sendai viral vector having G69E, T116A, and A183S mutations in the M protein, A262T, G264R, and K461G mutations in the HN protein, L511F mutation in the P protein, and N1197S and K1795E mutations in the L protein. The Sendai viral vector including KLF RNA and not including OCT RNA and SOX RNA is less temperature-sensitive than, for example, a Sendai viral vector into which the TS7, TS12, TS13, TS14, or TS15 mutation is introduced and can express the KLF gene at a predetermined temperature or higher.

The Sendai viral vector including KLF RNA and not including OCT RNA and SOX RNA is, for example, SeV18+KLF4/TSΔF.

When a plurality of types of Sendai viral vectors are introduced into cells, for example, a plurality of types of Sendai viral vectors are introduced into cells at the same time. Alternatively, within 48 hours, within 36 hours, within 24 hours, within 18 hours, within 12 hours, within 10 hours, within 8 hours, within 6 hours, within 3 hours, within 2 hours, or within 1 hour after a certain type of Sendai viral vector is introduced into cells, it is preferable to introduce all types of Sendai viral vectors into cells.

The multiplicity of infection (MOI) of the Sendai viral vector when cells are infected is, for example, 0.1 or more, 0.3 or more, 0.5 or more, 1.0 or more, 2.0 or more, 3.0 or more, 4.0 or more, or 5.0 or more. In addition, the MOI is, for example, 100 or less, 90 or less, 80 or less, 70 or less, 60 or less, 50 or less, 40 or less, 30 or less, 20 or less, 10 or less, or 5 or less.

The temperature at which cells are infected with a Sendai viral vector may be lower than a predetermined temperature at which the stability of the viral nucleic acid of the temperature-sensitive Sendai viral vector decreases, that is, a temperature at which the viral nucleic acid of the temperature-sensitive Sendai viral vector is stable, or a predetermined temperature or higher. When the Sendai viral vector is only a temperature-sensitive Sendai viral vector and does not include a temperature-insensitive Sendai viral vector, the temperature at which cells are infected with a Sendai viral vector is preferably a temperature that is lower than a predetermined temperature at which the stability of the viral nucleic acid of the temperature-sensitive Sendai viral vector decreases, that is, that is, a temperature at which the viral nucleic acid of the temperature-sensitive Sendai viral vector is stable.

Cells into which the reprogramming factor is introduced may be adherently cultured or suspension-cultured.

Somatic cells into which the reprogramming factor is introduced may be feeder-free cultured using a basement membrane matrix such as Matrigel (Corning), CELLstart (registered trademark, ThermoFisher), Laminin 511 (iMatrix-511, nippi), fibronectin, and vitrotin.

As the medium in which cells into which the reprogramming factor is introduced are cultured, a stem cell medium such as a human ES/iPS medium, for example, Primate ES Cell Medium (ReproCELL), can be used.

However, the stem cell medium is not limited thereto and various stem cell mediums can be used. For example, Primate ES Cell Medium, Reprostem, ReproFF, ReproFF2, ReproXF (Reprocell), mTeSR1, TeSR2, TeSRE8, ReproTeSR (STEMCELL Technologies), PluriSTEM (registered trademark) Human ES/iPS Medium (Merck), NutriStem (registered trademark) XF/FF Culture Medium for Human iPS and ES Cells, Pluriton reprogramming medium (Stemgent), PluriSTEM (registered trademark), Stemfit AK02N, Stemfit AK03 (Ajinomoto), ESC-Sure (registered trademark) serum and feeder free medium for hESC/iPS (Applied StemCell), L7 (registered trademark) hPSC Culture System (LONZA), and PluriQ (MTI-GlobalStem) may be used. The stem cell medium is put into an incubator, for example, a dish, a well, or a tube.

When cells are suspension-cultured or three-dimensionally cultured, for example, a gel medium is used. For example, the gel medium is prepared by adding gellan gum to a stem cell medium so that the final concentration is 0.001 mass % to 0.5 mass %, 0.005 mass % to 0.1 mass-, or 0.01 mass % to 0.05 mass %.

The gel medium may contain at least one polymer compound selected from the group consisting of gellan gum, hyaluronic acid, ramsan gum, diutan gum, xanthan gum, carrageenan, fucoidan, pectin, pectic acid, pectinic acid, heparan sulfate, heparin, heparitin sulfate, keratosulfate, chondroitin sulfate, dermatan sulfate, rhamnan sulfate, and salts thereof. In addition, the gel medium may contain methyl cellulose. When the gel medium contains methyl cellulose, aggregation between cells is further reduced.

Alternatively, the gel medium may contain a small amount of a temperature-sensitive gel selected from among poly(glycerol monomethacrylate) (PGMA), poly(2-hydroxypropyl methacrylate) (PHPMA), Poly(N-isopropylacrylamide) (PNIPAM), amine terminated, carboxylic acid terminated, maleimide terminated, N-hydroxysuccinimide (NHS) ester terminated, triethoxysilane terminated, Poly(N-isopropylacrylamide-co-acrylamide), Poly(N-isopropylacrylamide-co-acrylic acid), Poly(N-isopropylacrylamide-co-butylacrylate), Poly(N-isopropylacrylamide-co-methacrylic acid), Poly(N-isopropylacrylamide-co-methacrylic acid-co-octadecyl acrylate), and N-Isopropylacrylamide.

The gel medium may or may not contain a growth factor, for example, a basic fibroblast growth factor (bFGF). Alternatively, the gel medium may contain a growth factor such as bFGF at a low concentration of 400 μg/L or less, 40 μg/L or less, or 10 μg/L or less.

In addition, the gel medium may or may not contain TGF-β, and may contain TGF-β at a low concentration of 600 μg/L or less, 300 μg/L or less, or 100 μg/L or less.

The gel medium may not be stirred. In addition, the gel medium may not contain feeder cells.

The gel medium may contain at least one substance selected from the group consisting of cadherin, laminin, fibronectin, and vitronectin.

After cells are infected with a Sendai viral vector, for at least 2 days, or 2 days or more and 10 days or less, the cells may be cultured at a temperature lower than a predetermined temperature at which the stability of the viral nucleic acid of the temperature-sensitive Sendai viral vector decreases, that is, a temperature at which the viral nucleic acid of the temperature-sensitive Sendai viral vector is stable. Then, the cells may be cultured at a predetermined temperature or higher. When the cells are cultured at a predetermined temperature or higher, for example, the medium may be replaced once every two days.

After cells are infected with a Sendai viral vector, the cells may be cultured for at least 2 days, or 2 days or more and 10 days or less, for example, at a temperature of 4.0° C. or higher, 10° C. or higher, 15° C. or higher, 20° C. or higher, 25° C. or higher, 30° C. or higher, 31.0° C. or higher, 32.0° C. or higher, 33.0° C. or higher, 33.1° C. or higher, 33.2° C. or higher, 33.3° C. or higher, 33.4° C. or higher, 33.5° C. or higher, 33.6° C. or higher, 33.7° C. or higher, 33.8° C. or higher, or 33.9° C. or higher, lower than 37.0° C., lower than 36.9° C., lower than 36.8° C., lower than 36.7° C., lower than 36.6° C., lower than 36.5° C., 36.0° C. or lower, 35.0° C. or lower, or 34.0° C. or lower. Then, the temperature is raised, and the cells may be cultured at a temperature of 36.5° C. or higher, 36.6° C. or higher, 36.7° C. or higher, 36.8° C. or higher, 36.9° C. or higher, or 37.0° C. or higher, and 40.0° C. or lower, 39.0° C. or lower, or 38.0° C. or lower. The temperature may be raised once or stepwise. After the temperature is raised, when the cells are cultured, for example, the medium may be replaced once every two days.

After cells are infected with a Sendai viral vector, until stem-cell-like colonies begin to appear, the cells may be cultured at a temperature lower than a predetermined temperature at which the stability of the viral nucleic acid of the temperature-sensitive Sendai viral vector decreases, that is, a temperature at which the viral nucleic acid of the temperature-sensitive Sendai viral vector is stable. After the stem-cell-like colonies begin to appear, the cells may be cultured at a predetermined temperature or higher. When the cells are cultured at a predetermined temperature or higher, for example, the medium may be replaced once every two days.

After cells are infected with a Sendai viral vector, until stem-cell-like colonies begin to appear, the cells may be cultured at a temperature of, for example, 4.0° C. or higher, 10° C. or higher, 15° C. or higher, 20° C. or higher, 25° C. or higher, 30° C. or higher, 31.0° C. or higher, 32.0° C. or higher, 33.0° C. or higher, 33.1° C. or higher, 33.2° C. or higher, 33.3° C. or higher, 33.4° C. or higher, 33.5° C. or higher, 33.6° C. or higher, 33.7° C. or higher, 33.8° C. or higher, or 33.9° C. or higher, and lower than 37.0° C., lower than 36.9° C., lower than 36.8° C., lower than 36.7° C., lower than 36.6° C., lower than 36.5° C., 36.0° C. or lower, 35.0° C. or lower, or 34.0° C. or lower. After the stem-cell-like colonies begin to appear, the temperature is raised, and the cells may be cultured at a temperature of 36.5° C. or higher, 36.6° C. or higher, 36.7° C. or higher, 36.8° C. or higher, 36.9° C. or higher, or 37.0° C. or higher, and 40.0° C. or lower, 39.0° C. or lower, or 38.0° C. or lower. The temperature may be raised once or stepwise. After the temperature is raised, when the cells are cultured, for example, the medium may be replaced once every two days.

After a reprogramming factor is introduced into cells and the cells are cultured, cells into which the reprogramming factor is introduced are recovered, and at least some of the recovered and mixed cells are seeded and passaged in a medium, which is performed at least once. In the passage, clones of cells into which the reprogramming factor is introduced may be mixed. In the passage, different clones of cells into which the reprogramming factor is introduced may be mixed. Then, cells into which the reprogramming factor is introduced are recovered and at least some of the recovered and mixed cells are seeded and passaged in a medium, which may be performed a plurality of times. Until stem cells are derived, cells into which the reprogramming factor is introduced may be recovered, and at least some of the recovered and mixed cells may be seeded and passaged in a medium. Here, all the recovered and mixed cells may be seeded in a medium.

Here, recovering cells into which the reprogramming factor is introduced and seeding and passaging at least some of the recovered and mixed cells in a medium refers to, for example, passaging cells into which the reprogramming factor is introduced without distinguishing them according to the gene expression state. For example, during passage, cells into which the reprogramming factor is introduced may be seeded in the same incubator without distinguishing them according to the gene expression state. Alternatively, recovering cells into which the reprogramming factor is introduced and seeding and passaging at least some of the recovered and mixed cells in a medium refers to, for example, passaging cells into which the reprogramming factor is introduced without distinguishing them according to the degree of reprogramming. For example, during passage, cells into which the reprogramming factor is introduced may be seeded in the same incubator without distinguishing them according to the degree of reprogramming.

Alternatively, recovering cells into which the reprogramming factor is introduced and seeding and passaging at least some of the recovered and mixed cells in a medium refers to, for example, passaging cells into which the reprogramming factor is introduced without distinguishing them according to the form. For example, during passage, cells into which the reprogramming factor is introduced may be seeded in the same incubator without distinguishing them according to the form. Alternatively, recovering cells into which the reprogramming factor is introduced and seeding and passaging at least some of the recovered and mixed cells in a medium refers to, for example, passaging cells into which the reprogramming factor is introduced without distinguishing them according to the size. For example, during passage, cells into which the reprogramming factor is introduced may be seeded in the same incubator without distinguishing them according to the size.

In addition, alternatively, recovering cells into which the reprogramming factor is introduced and seeding and passaging at least some of the recovered and mixed cells in a medium refers to passaging without cloning cells into which the reprogramming factor is introduced. For example, when passaging is performed without cloning, it is not necessary to pick up colonies formed by cells into which the reprogramming factor is introduced. For example, when passaging is performed without cloning, a plurality of colonies formed by cells into which the reprogramming factor is introduced may not be isolated from each other. For example, during passage, cells forming a plurality of different colonies may be mixed and seeded in the same incubator. In addition, for example, when passaging is performed without cloning, it is not necessary to clone a single colony formed by cells into which the reprogramming factor is introduced. For example, during passage, colonies may be mixed and seeded in the same incubator.

For example, when cells into which the reprogramming factor is introduced are adherently cultured, cells that are adherently cultured may be recover, and at least some of the recovered and mixed cells may be seeded and passaged in a medium. For example, during passage, all cells may be separated from the incubator, and at least some of the separated and mixed cells may be seeded in the same incubator. For example, all cells may be separated from the incubator with a separation solution and all separated and mixed cells may be passaged. For example, cells that do not form colonies may be passaged. When cells into which the reprogramming factor is introduced are suspension-cultured, all suspension-cultured cells may be passaged.

When cells into which the reprogramming factor is introduced are passaged, the cells are seeded in a medium or incubator at a low concentration. Here, the low concentration is, for example, 1 cell/cm² or more, 0.25×10⁴ cells/cm² or less, 1.25×10³ cells/cm² or less, 0.25×10³ cells/cm² or less, 0.25×10² cells/cm² or less, or 0.25×10¹ cells/cm² or less. Alternatively, the low concentration is a concentration at which 10 cells or less, 9 cells or less, 8 cells or less, 7 cells or less, 6 cells or less, 5 cells or less, 4 cells or less, 3 cells or less, or 2 cells or less can come into contact with each other and 11 cells or more do not come into contact with each other. Here, there may be a plurality of cell masses in which 10 cells or less come into contact with each other. Alternatively, the state in which the entire bottom surface of the cell container is covered with cells is regarded as 100% confluency, and the low concentration is 5% or less confluency, 4% or less confluency, 3% or less confluency, 2- or less confluency, 1% or less confluency, 0.5% or less confluency, 0.1% or less confluency, 0.05% or less confluency, or 0.01% or less confluency. In addition, alternatively, the low concentration is, for example, a concentration at which single cells do not come into contact with each other in the seeded cells. For example, single cells may be seeded in wells of a well plate. The well plate may be a 12-well plate or a 96-well plate. According to the findings of the inventors, when cells into which the reprogramming factor is introduced are passaged, cells are seeded in a medium at a low concentration, and thus the residual Sendai virus in pluripotent stem cells induced from cells can be minimized. The proportion of cells in which the Sendai virus remains among the induced pluripotent stem cells is, for example, 4% or less, 3% or less, 2% or less, 1% or less, 0.5% or less, or 0%.

When the temperature-sensitive Sendai viral vector is used, after passage, cells may be cultured at a predetermined temperature or higher at which the stability of the viral nucleic acid of the temperature-sensitive Sendai viral vector decreases. After passage, for example, cells may be cultured at a temperature of 36.5° C. or higher and lower than 38.0° C. After passage, for example, cells are cultured at a temperature of 36.5° C. or higher and lower than 38.0° C. until intercellular adhesion starts, and after intercellular adhesion starts, until intercellular adhesion starts, cells may be cultured at a higher temperature, for example, at a temperature of 37.5° C. or higher, 42.0° C. or lower, 41.5° C. or lower, 41.0° C. or lower, 40.5° C. or lower, or 40.0° C. or lower. After passage, before intercellular adhesion starts, cells may be cultured at a temperature of 37.5° C. or higher, 42.0° C. or lower, 41.5° C. or lower, 41.0° C. or lower, 40.5° C. or lower, or 40.0° C. or lower.

Cells into which the reprogramming factor is introduced may be cultured and passaged in a closed incubator. In the closed incubator, for example, gases, viruses, microorganisms and impurities are not exchanged with the outside. In addition, cells into which the reprogramming factor is introduced may be expansion-cultured in two-dimensional culture or may be expansion-cultured in three-dimensional culture.

After cells into which the reprogramming factor is introduced are induced to pluripotent stem cells and pluripotent stem cells are derived, all adherently cultured cells may be cryopreserved as pluripotent stem cells. For example, all cells separated from the incubator with a separation solution may be cryopreserved as pluripotent stem cells. In addition, after cells into which the reprogramming factor is introduced are induced to pluripotent stem cells, all suspension-cultured cells may be cryopreserved as pluripotent stem cells.

The induced pluripotent stem cells can form flat colonies similar to ES cells and express alkaline phosphatase. The induced pluripotent stem cells can express undifferentiated cell markers Nanog, OCT4, SOX2 and the like. The induced pluripotent stem cells can express TERT. The induced pluripotent stem cells can exhibit telomerase activity.

In addition, determination of whether cells are induced to pluripotent stem cells may be performed by analyzing whether at least one surface marker selected from among cell surface markers TRA-1-60, TRA-1-81, SSEA-1, and SSEA5 which indicate undifferentiation, with a cyto flow meter, is positive. TRA-1-60 is an antigen specific for iPS/ES cells. Since iPS cells can be produced only from TRA-1-60 positive fractions, TRA-1-60 positive cells are considered to be the species of iPS cells.

The induced pluripotent stem cells may be induced to somatic cells in a state different from the state of the pluripotent stem cells. Examples of somatic cells include nerve cells, omental epithelial cells, hepatocytes, β cells, kidney cells, dental pulp stem cells, mesenchymal stem cells, somatic prestem cells, keratinocytes, dermal papilla cells, oral epithelial cells, cartilage cells, muscle cells, vascular cells, epithelial cells, cardiomyocytes, blood cells, and immune cells. Examples of blood cells include erythroblasts, red blood cells, megakaryocytes, and platelets. Examples of immune cells include monocytes, neutrophilic leukocytes, eosinophilic leukocytes, basophil leukocytes, B cells, T cells, NK cells, and NKT cells. The induced stem cells may be differentiated into the endoderm, the mesoderm, or the ectoderm. Stem cells may form embryoid bodies, organoids, and spheres.

Examples of factors that induce cells into nervous system cells include ASCL family, DLX family, MYT family, NeuroD family, SOX family, and NGN family. Examples of ASCL family include ASCL1. Examples of DLX family include DLX2. Examples of MYT family include MYT1L. Examples of NGN family include NGN2. Examples of nervous system cells include nerve cells, neural stem cells and neural progenitor cells. Examples of nerve cells include inhibitory nerve cells, excitatory nerve cells, dopamin-producing nerve cells, cranial nerves, intervening nerves, and optic nerves. Alternatively, nervous system cells may be motor nerve cells, oligodendrocyte progenitor cells, astrocytes, oligodendrocytes or the like.

Examples of factors that induce cells into cardiomyocytes include GATA family, MEF family, TBX family, MYOCD family, MESP family, and miR-133 family. Examples of GATA family include GATA4A. Examples of MEF family include MEF2C. Examples of TBX family include TBX5. Examples of MESP family include MESP1.

Here, in the present disclosure, induction refers to reprogramming, initialization, transformation, transdifferentiation (Transdifferentiation or Lineage reprogramming), differentiation induction, cell fate change (Cell fate reprogramming) or the like.

After the derived pluripotent stem cells are treated so that they are induced to somatic cells different from the pluripotent stem cells, the induced cells may be cloned.

After the derived pluripotent stem cells are subjected to a gene editing process, gene-edited cells may be cloned.

Here, without passaging any cells into which the reprogramming factor is introduced, cells into which the reprogramming factor is introduced may be induced to somatic cells different from the pluripotent stem cells. A method for introducing a reprogramming factor into cells is as described above. For example, without passaging any cells into which the reprogramming factor is introduced, a differentiation-inducing factor may be introduced into cells into which the reprogramming factor is introduced, and the cells into which the reprogramming factor is introduced may be induced to somatic cells different from the pluripotent stem cells. Alternatively, without passaging any cells into which the reprogramming factor is introduced, a hormone or chemical substance may be applied to cells into which the reprogramming factor is introduced, and the cells into which the reprogramming factor is introduced may be induced to somatic cells different from the pluripotent stem cells. In this case, cells into which the reprogramming factor is introduced are induced to somatic cells without being cloned. The induced somatic cells are as described above.

### EXAMPLES

### Example 1 and Comparative Example 1

A dish coated with laminin 511 was used as a dish for inducing pluripotent stem cells. In addition, human peripheral blood mononuclear cells were suspended in a blood medium, the number of mononuclear cells was measured using a blood cell counting chamber, and the number of mononuclear cells in the blood medium was adjusted. Then, the mononuclear cells were two-dimensionally cultured on the dish for inducing pluripotent stem cells at 37° C. for 1 to 7 days.

SeV(PM)hKOS/TS12ΔF and SeV(HNL)hC-Myc/TS15ΔF were added to two-dimensionally cultured mononuclear cells so that the MOI was 5, the dish for inducing pluripotent stem cells was accommodated in an incubator at 34° C., and the cells were cultured. Two days after infection, the blood medium was replaced with an iPS cell medium. Then, the medium was replaced once every two days using the iPS cell medium. Along the way, the temperature was gradually raised to 37° C., 38° C.

8 days after infection, stem-cell-like cell masses generated. On the 14th day after infection, almost all cells become TRA1-60 positive cells, and showed iPS-cell-like morphology. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In Example 1, during the first passage, all cells were separated from the well plate, and the separated and mixed cells were seeded in the next well plate without distinguishing. On the other hand, in Comparative Example 1, during the first passage, colonies were picked and cloned. Here, in both Example 1 and Comparative Example 1, during passage, cells were seeded so that 11 or more cells did not come into contact with each other.

Next, the well-dish was accommodated in an incubator at 37° C., and cells were two-dimensionally cultured. After the cells began to divide, the culture temperature was raised to 38° C. Then, in both Example 1 and Comparative Example 1, all cells were recovered whenever the cells had 60% to 80% confluency, and at least some of the recovered and mixed cells were seeded and passaged in a medium. From the second passage onward, the cells were seeded in the well plate so that the concentration was 0.25×10⁴ cells/cm² or less. In this case also, 11 or more cells did not come into contact with each other.

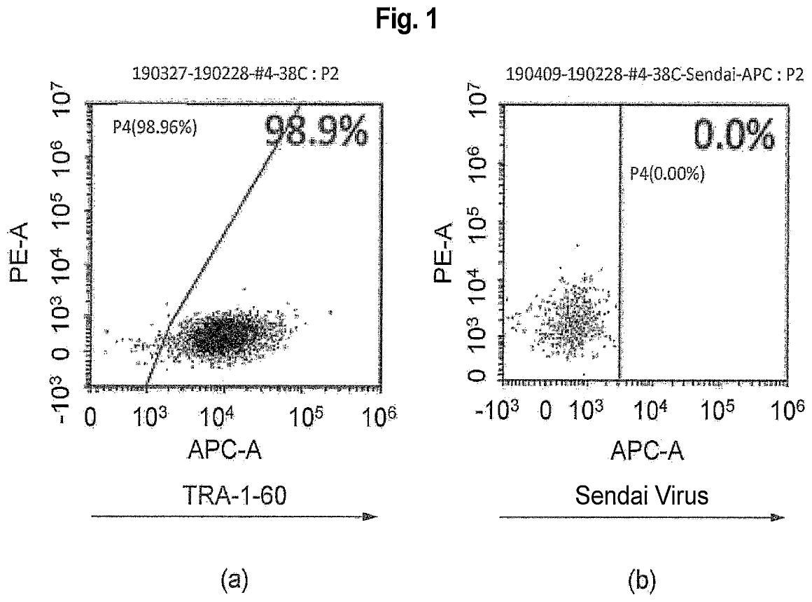

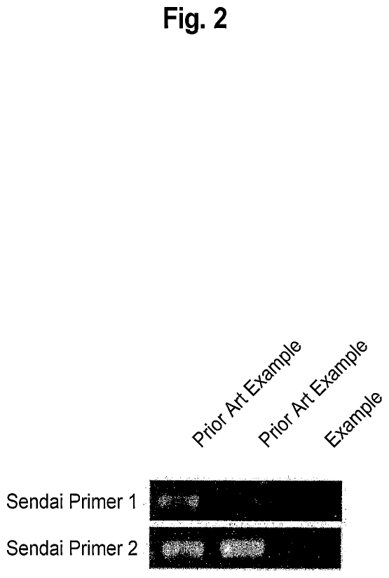

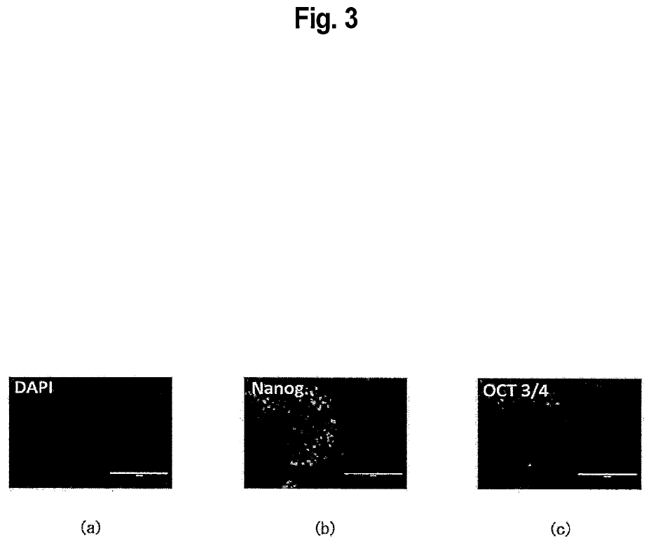

As shown in FIG. 1, when the cells that had been passaged only once were stained with anti-Sendai virus antibodies and the Sendai virus remaining in the cells according to Example 1 was evaluated with a flow cytometer, the Sendai virus in the cells disappeared. As shown in FIG. 2, the Sendai virus remaining in the cells according to Example 1 was not detected by PCR. As in the prior art, when cells were seeded at a high concentration at which 11 or more cells were adhered to each other, the Sendai virus remained in the cells. FIG. 3 shows immunostaining images of the obtained TRA1-60 positive cells.

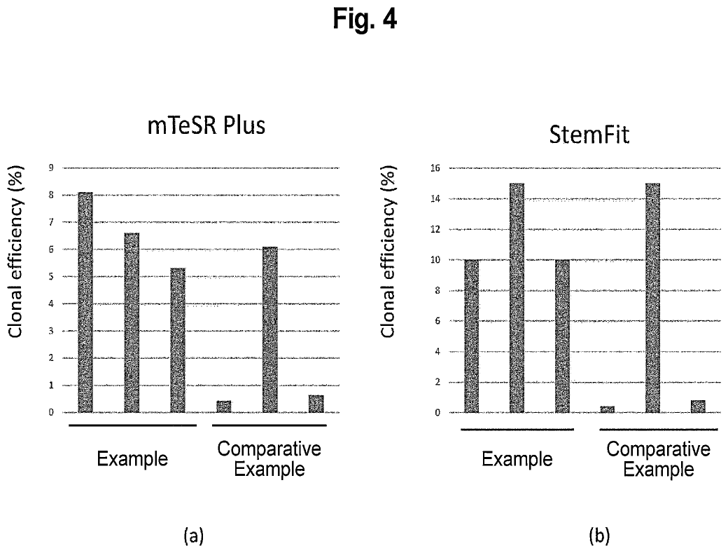

In addition, the number of colonies formed 5 days after the Sendai virus in the cells disappeared was counted. In addition, the clonal efficiency was calculated by dividing the number of colonies by the number of seeded cells. FIG. 4 shows the results of three tests. When all cells were recovered using mTeSR Plus as a medium during the first passage, and some of the recovered and mixed cells were seeded and passaged in the medium, the clonal efficiency was about 5% to about 8%, with a small variation. When colonies were picked and cloned using mTeSR Plus as a medium during the first passage, the clonal efficiency may be less than 1% or about 6%, there was a variation in the clonal efficiency. When all cells were recovered using StemFit as a medium during the first passage, and some of the recovered and mixed cells were seeded and passaged in the medium, the clonal efficiency was about 10% to about 15%, with a small variation. When colonies were picked and cloned using StemFit as a medium during the first passage, the clonal efficiency may be less than 1% or about 16%, there was a variation in the clonal efficiency.

Therefore, when all cells into which the reprogramming factor was introduced were recovered during the first passage and at least some of the recovered and mixed cells were seeded and passaged in a medium, it was shown that the clonal efficiency was high and stable.

### Example 2 and Comparative Example 2

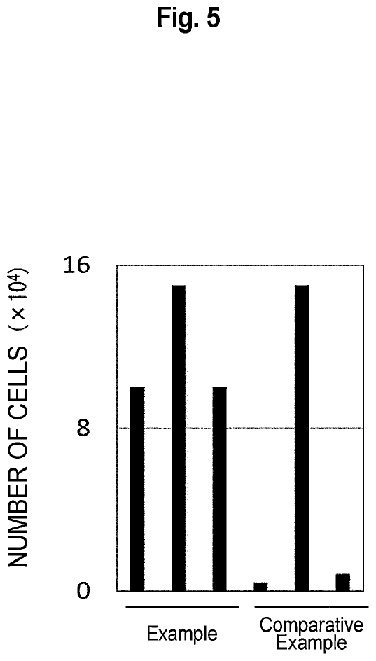

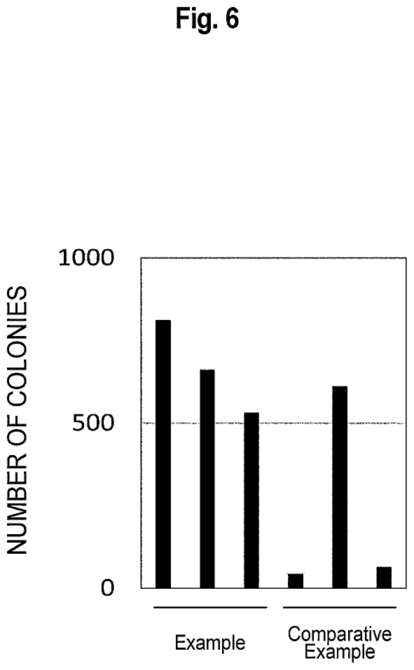

The iPS-cell-like cells obtained by passage in the same manner as in Example 1 and Comparative Example 1 were separated and dissociated. Next, about 1×10⁵ cells were cryopreserved using STEM-CELLBANKER (registered trademark, Takara). Then, the frozen cells were melted, about 1×10⁴ cells were seeded in the well, and the cells were cultured and proliferated. The number of cells and the number of colonies 7 days after seeding were measured. FIG. 5 and FIG. 6 show the results of three tests.

As shown in FIG. 5, when all cells were recovered and some of the recovered and mixed cells were seeded and passaged in a medium during the first passage, the number of cells cultured for 7 days after freezing and melting was about 10×10⁴ to about 15×10⁴, with a small variation. On the other hand, in cells cloned by picking colonies during the first passage, the number of cells cultured for 7 days after freezing and melting was about 0.4×10⁴ to about 15×10⁴ cells, with a large variation. Therefore, when all cells into which the reprogramming factor was introduced were recovered during the first passage and at least some of the recovered and mixed cells were seeded and passaged in a medium, it was shown that the proliferation rate of the cells after freezing and melting was high and stable.

As shown in FIG. 6, when all cells were recovered during the first passage and at least some of the recovered and mixed cells were seeded and passaged in a medium, the number of colonies of cells cultured for 7 days after freezing and melting was about 500 to about 800 colonies, with a small variation. On the other hand, in cells cloned by picking colonies during the first passage, the number of colonies of cells cultured for 7 days after freezing and melting was about 30 to about 600 colonies, with a large variation. Therefore, when all cells into which the reprogramming factor was introduced were recovered during the first passage and at least some of the recovered and mixed cells were seeded and passaged in a medium, it was shown that the colony forming rate of cells after freezing and melting was high and stable.

### Example 3 and Comparative Example 3

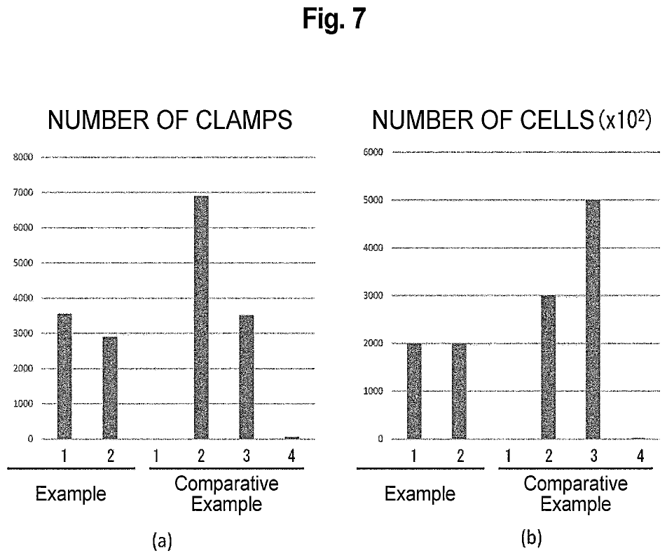

iPS-cell-like cells obtained by passage in the same manner as in Example 1 and Comparative Example 1 were separated, 2.5×10⁵ cells were suspended in a gel medium, the cells were three-dimensionally cultured, and clamps were formed in the cells. The number of clamps and the number of cells 13 days after the cells were seeded in the gel medium were measured. FIG. 7 shows the results obtained by testing the cells obtained in Example 1 twice and the cells obtained in Comparative Example 1 4 times.

When all cells were recovered during the first passage and some of the recovered and mixed cells were seeded and passaged in a medium, the cells were three-dimensionally cultured, about 3,000 clamps were formed, and the variation between tests was small. In addition, when all cells were recovered during the first passage and some of the recovered and mixed cells were seeded and passaged in a medium, the cells were three-dimensionally cultured and about 2,000×10² cells were obtained, and the variation between tests was small. On the other hand, in cells cloned by picking colonies during the first passage, about 7,000 clamps were formed in some tests, but cells died and formed almost no clamps in some tests, and the variation between tests was large. In addition, cells cloned by picking colonies during the first passage were three-dimensionally cultured and about 5,000×10² cells were obtained in some tests, but cells died and the number of cells was almost 0 in some tests, and the variation between tests was large.

Therefore, when all cells into which the reprogramming factor was introduced were recovered during the first passage and at least some of the recovered and mixed cells were seeded and passaged in a medium, it was shown that the proliferation rate and the clamp forming ability of cells in the subsequent three-dimensional culture were improved and stable.

### Example 4 and Comparative Example 4

iPS-cell-like cells obtained by passage in the same manner as in Example 1 and Comparative Example 1 were separated, and the iPS-cell-like cells were differentiated into cardiomyocytes using a cardiomyocyte differentiation induction kit (PSC Cardiomyocyte Differentiation Kit, Gibco, registered trademark).

Specifically, in a 12-well plate whose bottom surface was treated with a basement membrane matrix (Corning Matrigel, registered trademark), about 2×10⁴ to about 6×10⁴ iPS-cell-like cells were seeded, and the cells were cultured using mTeSR1 as a medium. Two days after iPS-cell-like cells were seeded, the medium was replaced with a Cardiomyocyte Differentiation Medium A. Two days later, the medium was replaced with a Cardiomyocyte Differentiation Medium B. Two days later, the medium was replaced with a Cardiomyocyte Maintenance Medium. Then, every two days up to the 22nd day, the cells were cultured after the iPS-cell-like cells were seeded while the medium was replaced with a Cardiomyocyte Maintenance Medium.

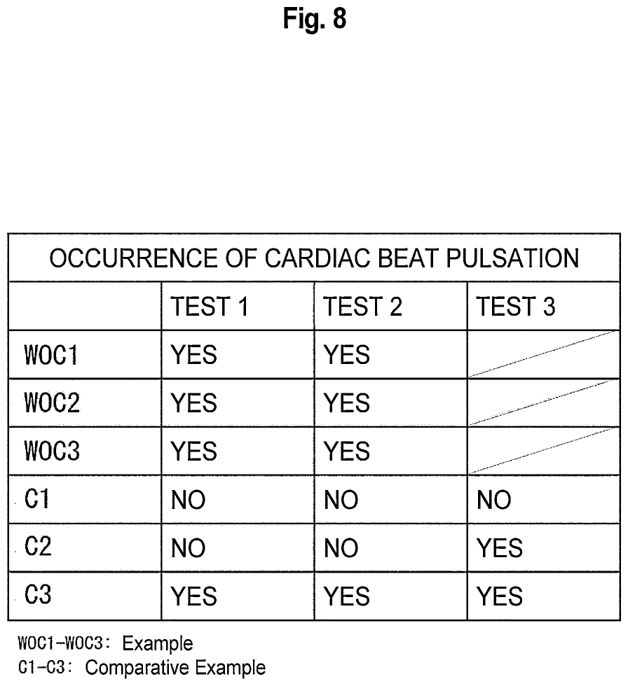

As a result, as shown in FIG. 8, after a reprogramming factor was introduced before cells were induced to differentiate into cardiomyocytes, all cells were recovered during the first passage, and cells obtained by seeding and passaging some of the recovered and mixed cells in a medium showed pulsation on the 22nd day in all the tests. On the other hand, after a reprogramming factor was introduced before cells were induced to differentiate into cardiomyocytes, cells cloned by picking colonies during the first passage showed pulsation on the 22nd day only in less than half of the tests.

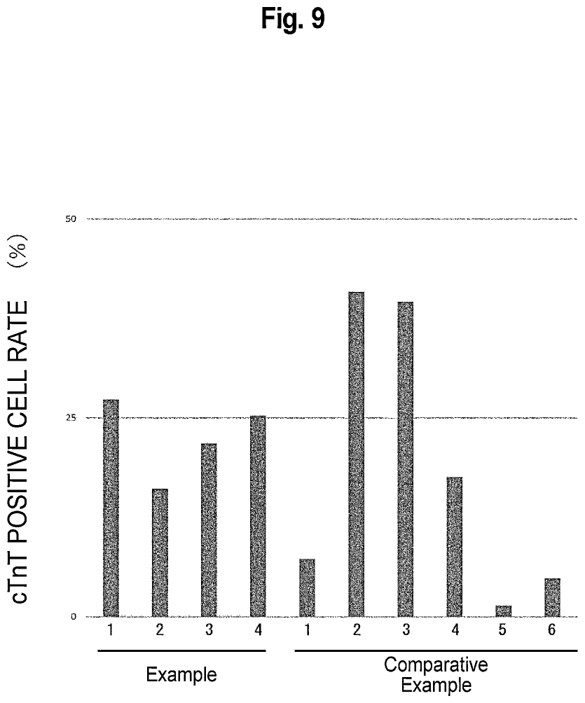

In addition, when the positive rate of cardiac troponin T (cTnT), which is a marker of cardiomyocytes, was examined by FACS, as shown in FIG. 9, after a reprogramming factor was introduced before cells were induced to differentiate into cardiomyocytes, all cells were recovered during the first passage, and cells obtained by seeding and passaging some of the recovered and mixed cells in a medium were stable with a cTnT positive rate of around 20%. On the other hand, after a reprogramming factor was introduced before cells were induced to differentiate into cardiomyocytes, cells cloned by picking colonies during the first passage had a large variation in the cTnT positive rate of about 1% to about 37%.

Therefore, when all cells into which the reprogramming factor was introduced were recovered during the first passage, at least some of the recovered and mixed cells were seeded and passaged in a medium, and stem cells were derived, it was shown that an ability to induce differentiation into somatic cells such as cardiomyocytes was strong and stable thereafter.

### Example 5 and Comparative Example 5

The iPS-cell-like cells obtained by passage in the same manner as in Example 1 and Comparative Example 1 were separated, and 1.5×10⁵ cells were seeded and cultured in a round-bottomed cell culture plate having a size suitable for cell mass formation (Kuraray, RB 500 400 NA 6).

For the medium, an 8GMK medium (8, KnockOut Serum Replacement (Life Technologies)) to which ALK-4, -5, -7 selective inhibitor for TGF-β1 activin receptor-like kinase (ALK) (500 nmol/L, A-83-01, Stemgent) and a membrane permeable inhibitor (100 nmol/L, LDN193189, Stemgent) for BMP Type I receptor (ALK2, ALK3) were added, 1% non-essential amino acid (NEAA, Life Technologies), and 1% sodium pyruvate (Sigma), 100 nmol/L 2-mercaptoethanol (2-ME, Life Technologies) were used. It was known that pluripotent stem cells cultured in the presence of the above inhibitor were inducted to differentiate into neural progenitor cells.

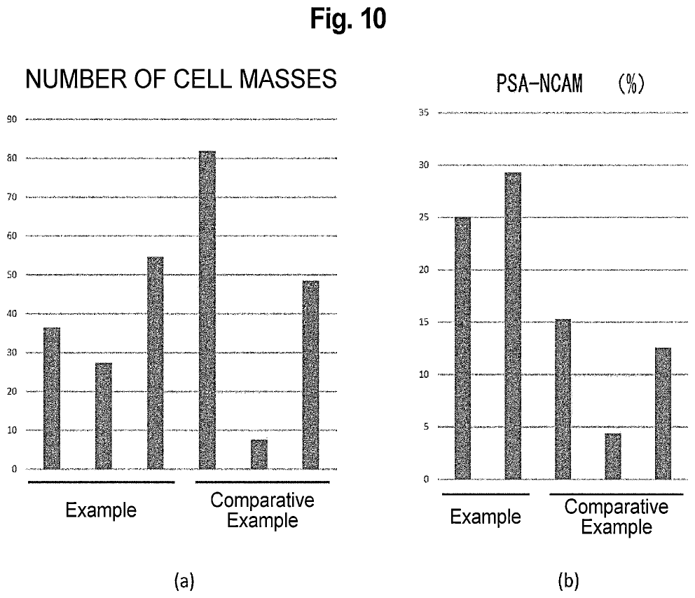

The medium was replaced 5 days, 8 days, and 11 days after the cells were seeded. After 14 days, when the number of cell masses was measured, as shown in FIG. 10, after a reprogramming factor was introduced before cells were induced to differentiate into neural progenitor cells, all cells were recovered during the first passage, and cells obtained by seeding and passaging at least some of the recovered and mixed cells in a medium formed about 25 to about 55 neuropheres, with a small variation. On the other hand, after a reprogramming factor was introduced before cells were induced to differentiate into neural progenitor cells, cells cloned by picking colonies during the first passage formed about 10 to about 80 cell masses, with a large variation.

In addition, after 14 days, cells were recovered in a tube and centrifuged, cell masses were decomposed into single cells with a cell dissociator (TrypLE Select, ThermoFisher, registered trademark), and the number of cells was measured, and cells were then immunostained using PSA-NCAM antibodies which are antibodies that detect polysialization molecules of nerve cell adhesion molecules (N-CAM), and the positive rate of PSA-NCAM was analyzed by flow cytometry. As a result, as shown in FIG. 10, after a reprogramming factor was introduced before cells were induced to differentiate into neural progenitor cells, all cells were recovered during the first passage, and cells obtained by seeding and passaging at least some of the recovered and mixed cells in a medium had a positive rate of PSA-NCAM of about 25% to about 30%, with a small variation. On the other hand, after a reprogramming factor was introduced before cells were induced to differentiate into neural progenitor cells, cells cloned by picking colonies during the first passage had a positive rate of PSA-NCAM of about 5% to about 15%, with a large variation.

Therefore, when all cells into which the reprogramming factor was introduced were recovered during the first passage, at least some of the recovered and mixed cells were seeded and passaged in a medium, and stem cells were derived, it was shown that an ability to induce differentiation into somatic cells such as nerve cells was strong and stable thereafter.

### Example 6 and Comparative Example 6

The iPS-cell-like cells obtained by passage in the same manner as in Example 1 and Comparative Example 1 were separated, and 1×10⁵ cells were seeded and cultured in a non-adhesive dish. Regarding the medium, a human ES cell medium to which no bFGF was added was used. In addition, the medium was replaced once every two days. 9 days after seeding, the formed embryoid bodies (EB) were seeded again in a gelatin-coated 6-well plate. Then, the medium was replaced once every two days, and the cells were recovered by a trypsin treatment on the 24th day after seeding again. The recovered cells were immobilized with 4%, paraformaldehyde, the cells were stained using SOX1 antibodies, OTC2 antibodies, HAND1 antibodies, and SOX17 antibodies, and the stained cells were analyzed using a flow cytometer. Here, SOX1 and OTX2 are ectoderm markers, HAND1 is a mesoderm marker, and SOX17 is an endoderm marker.

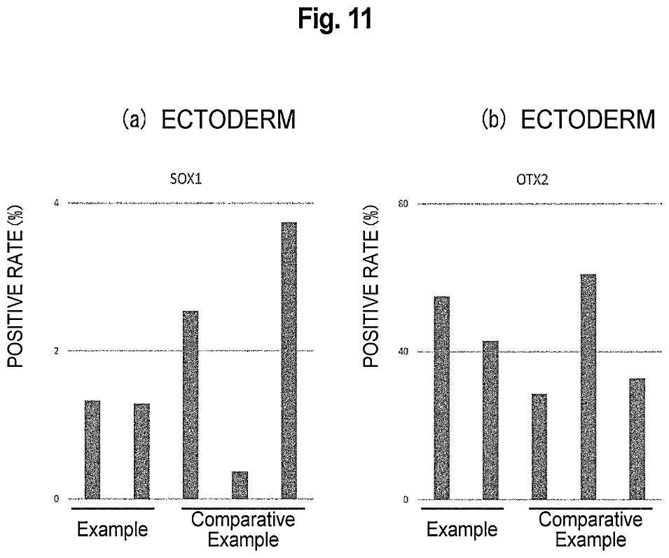

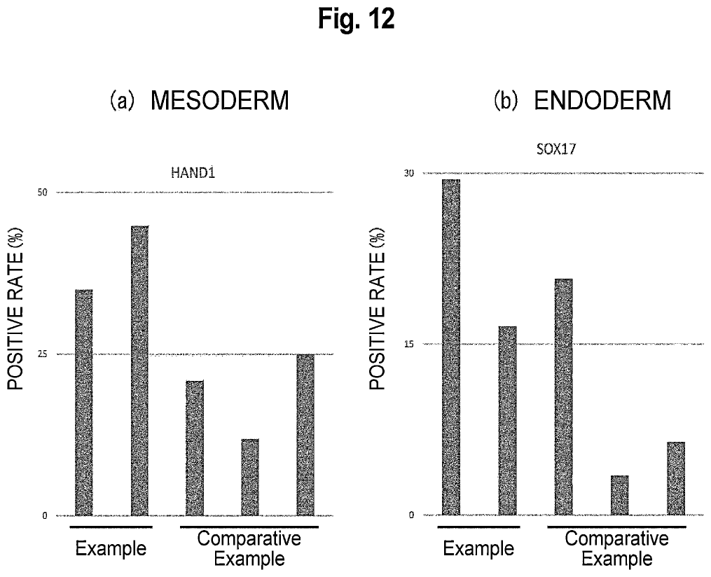

As a result, as shown in FIG. 11 and FIG. 12, after a reprogramming factor was introduced, all cells were recovered during the first passage, and cells obtained by seeding and passaging some of the recovered and mixed cells in a medium had a small variation in the positive rate of each marker. On the other hand, after a reprogramming factor was introduced, cells cloned by picking colonies during the first passage had a large variation in the positive rate of each marker. Therefore, when all cells into which the reprogramming factor was introduced were recovered during the first passage and at least some of the recovered and mixed cells were seeded and passaged in a medium, it was shown that an ability to differentiate into the endoderm, the mesoderm, and the ectoderm was strong and stable thereafter.

### Reference Example 1

SeV(PM)hKOS/TS12ΔF, SeV18+hKLF4/TSΔF, and SeV(HNL)hC-Myc/TS15ΔF were added to two-dimensionally cultured fibroblasts so that the MOI was 5, a dish for inducing pluripotent stem cells was accommodated in an incubator at 34° C., and the cells were cultured. Two days after infection, the blood medium was replaced with an iPS cell medium. Then, the medium was replaced once every two days using the iPS cell medium. On the 14th day after infection, the culture temperature was gradually raised to 37° C. and 38° C.

10 days after infection, stem-cell-like cell masses generated. On the 14th day after infection, almost all cells become TRA1-60 positive cells, and showed iPS-cell-like morphology. 14 days after infection, a TrypLE select as a cell-releasing agent was added to the dish, and a cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Next, the well-dish was accommodated in an incubator, and the cells were two-dimensionally cultured. After the cells began to divide, the culture temperature was set to 38° C. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other.

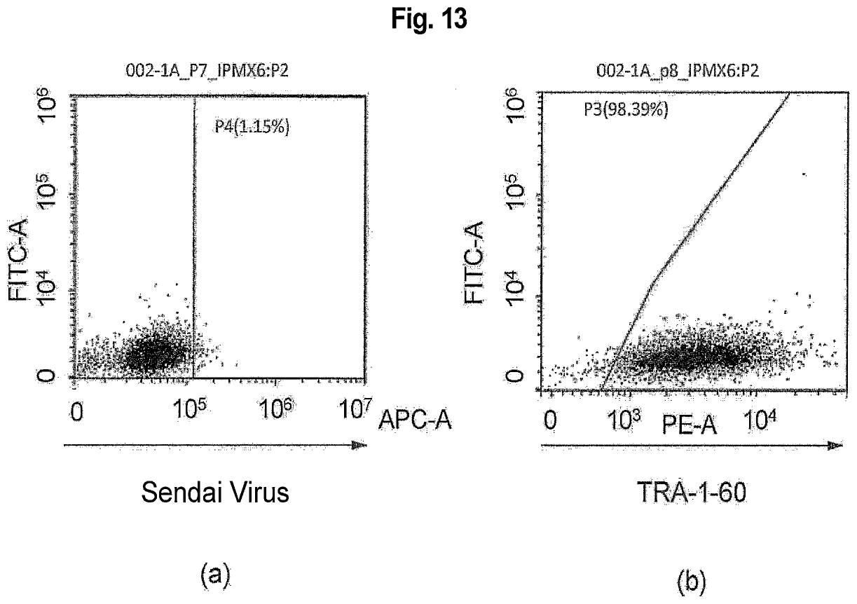

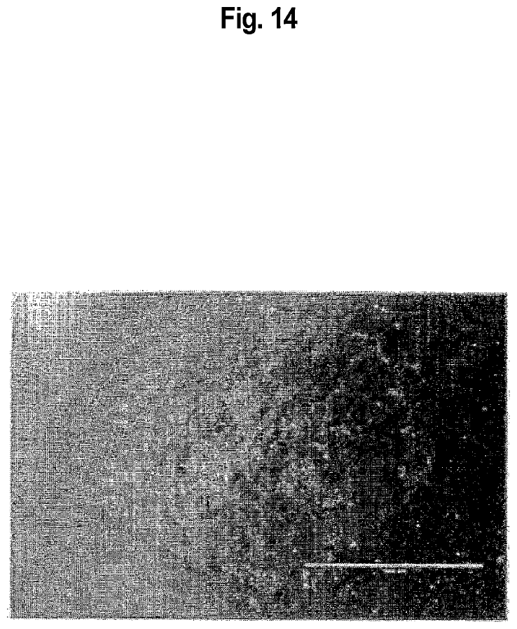

As shown in FIG. 13, when the cells that had been passaged only once were stained with anti-Sendai virus antibodies and the Sendai virus remaining in the cells was evaluated with a flow cytometer, the Sendai virus in the cells was almost disappeared. FIG. 14 shows an image of the obtained TRA1-60 positive cells.

### Reference Example 2

SeV(PM)hKOS/TS12ΔF, SeV18+hKLF4/TSΔF, and SeV(HNL)hC-Myc/TS15ΔF were added to two-dimensionally cultured mononuclear cells so that the MOI was 5, a dish for inducing pluripotent stem cells was accommodated in an incubator at 37° C. and the cells were cultured. Two days after infection, the blood medium was replaced with an iPS cell medium. Then, the medium was replaced once every two days using the iPS cell medium.

8 days after infection, stem-cell-like cell masses generated. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The concentration of the cell-containing solution was adjusted so that 11 or more cells adhered to each other, and the cells were seeded in a well plate for the first passage so that the concentration was higher than 0.25×10⁴ cells/cm². Next, the well-dish was accommodated in an incubator at 37° C., and cells were two-dimensionally cultured. After the cells began to divide, the culture temperature was raised to 38° C. Then, the cells were passaged so that the cells had 60% to 80% confluency. From the second passage to the fifth passage, 11 or more cells adhered to each other, and the cells were seeded in a well plate so that the concentration was higher than 0.25×10⁴ cells/cm². From the sixth passage onward, the cells were seeded in a well plate so that the concentration was 0.25×10⁴ cells/cm² or less. In this case, 11 or more cells did not come into contact with each other.

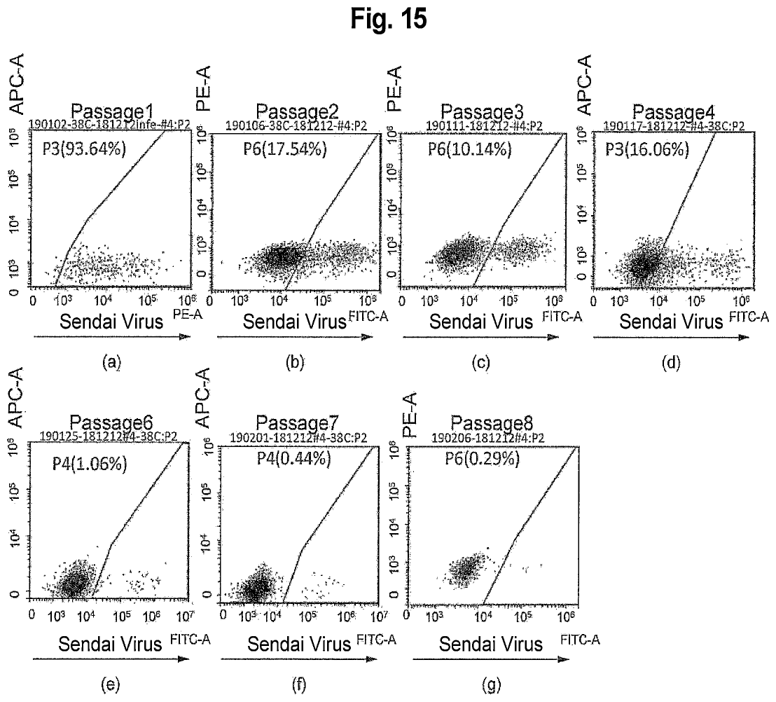

As shown in FIG. 15, when the cells were stained using anti-Sendai virus antibodies and the Sendai virus remaining in the cells was evaluated with a flow cytometer, the Sendai virus remained in the cells before the sixth passage. However, in the sixth passage onward in which the cells were seeded in a well plate so that the concentration was 0.25×10⁴ cells/cm² or less, the Sendai virus in the cells rapidly disappeared.

### Reference Example 3

CytoTune-iPS2.0 (registered trademark, ID Pharma), which is a Sendai viral vector kit, was prepared. CytoTune-iPS2.0 includes SeV(PM)hKOS/TS12ΔF, which is a temperature-sensitive Sendai viral vector that carries KLF4 genes, OCT3/4 genes, and SOX2 genes as reprogramming factors, SeV18+hKLF4/TSΔF, which is a temperature-sensitive Sendai viral vector that carries KLF4 as a reprogramming factor, and SeV(HNL)hC-Myc/TS15ΔF, which is a temperature-sensitive Sendai viral vector that carries c-MYC as a reprogramming factor.

SeV(PM)hKOS/TS12ΔF, SeV18+hKLF4/TSΔF, and SeV(HNL)hC-Myc/TS15ΔF were added to mononuclear cells so that the MOI was 5, a dish for inducing pluripotent stem cells was accommodated in an incubator at 37° C., and the cells were cultured. Two days after infection, the blood medium was replaced with an iPS cell medium (mTeSR Plus (STEMCELL Technologies) or StemFit (Ajinomoto)). Then, the medium was replaced once every two days using the iPS cell medium.

8 days after infection, stem-cell-like cell masses generated. On the 14th day after infection, almost all cells become TRA1-60 positive cells, and showed iPS-cell-like morphology. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other.

Next, the well-dish was accommodated in an incubator at 37° C., and cells were two-dimensionally cultured. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other.

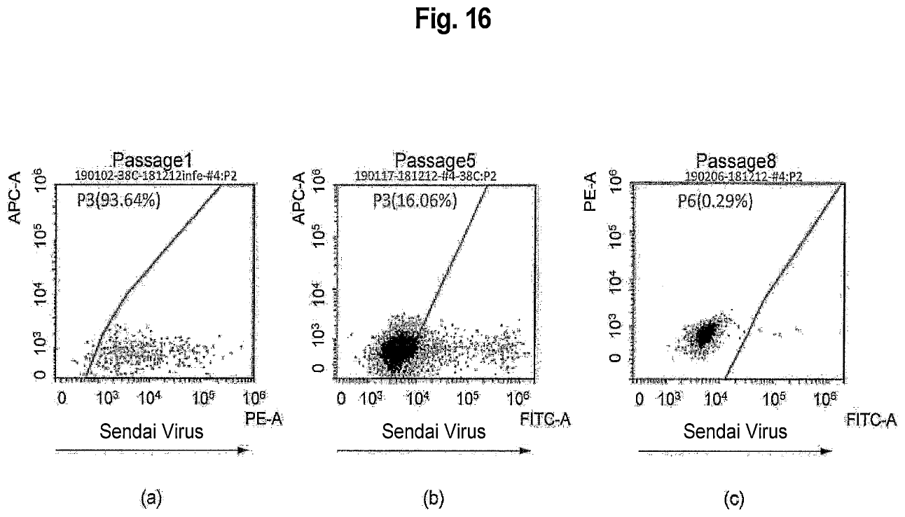

As shown in FIG. 16, when the cells were stained using anti-Sendai virus antibodies and the Sendai virus remaining in the cells was evaluated with a flow cytometer, at the 8th passage, the Sendai virus in the cells almost disappeared.

### Reference Example 4

SeV(PM)hKOS/TS12ΔF and SeV(HNL)hC-Myc/TS15ΔF were added to two-dimensionally cultured mononuclear cells so that the MOI was 5, the dish for inducing pluripotent stem cells was accommodated in an incubator at 34° C., and the cells were cultured. Two days after infection, the blood medium was replaced with an iPS cell medium. Then, the medium was replaced once every two days using the iPS cell medium. Along the way, the temperature was raised to 38° C.

8 days after infection, stem-cell-like cell masses generated. On the 14th day after infection, almost all cells become TRA1-60 positive cells, and showed iPS-cell-like morphology. 15 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Next, the well-dish was accommodated in an incubator at 38° C., and the cells were two-dimensionally cultured. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other.

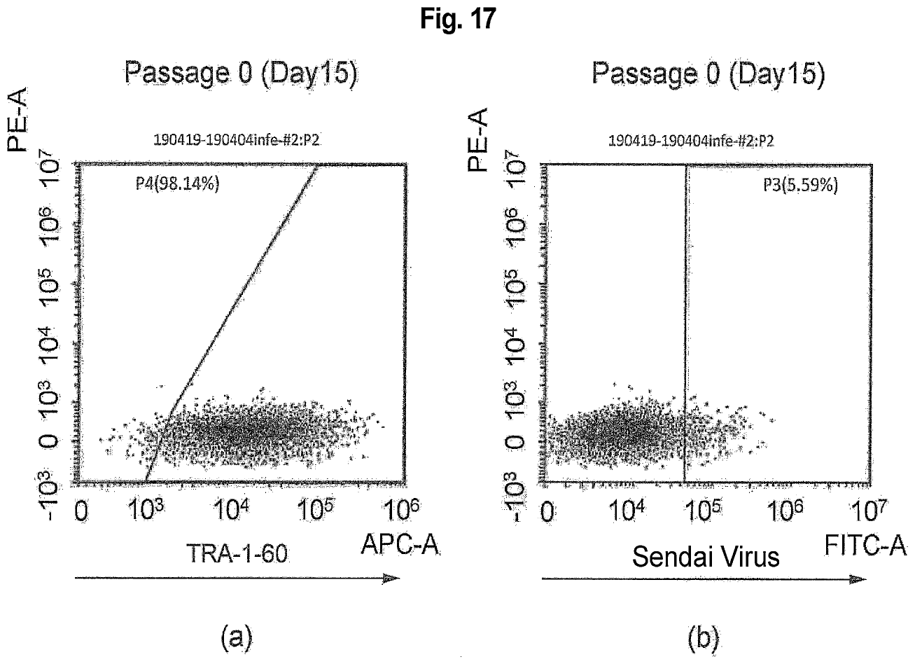

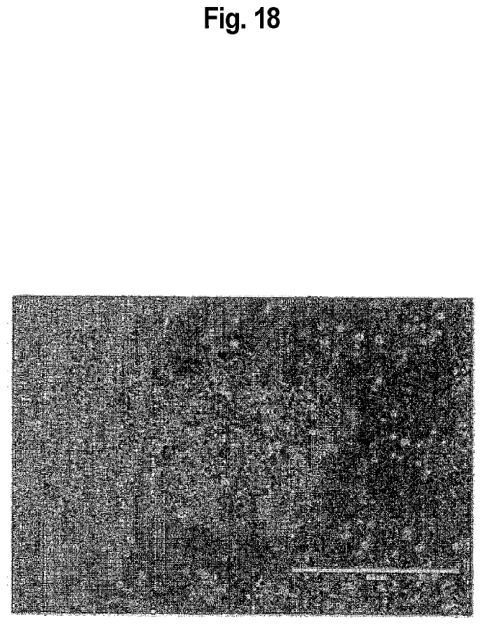

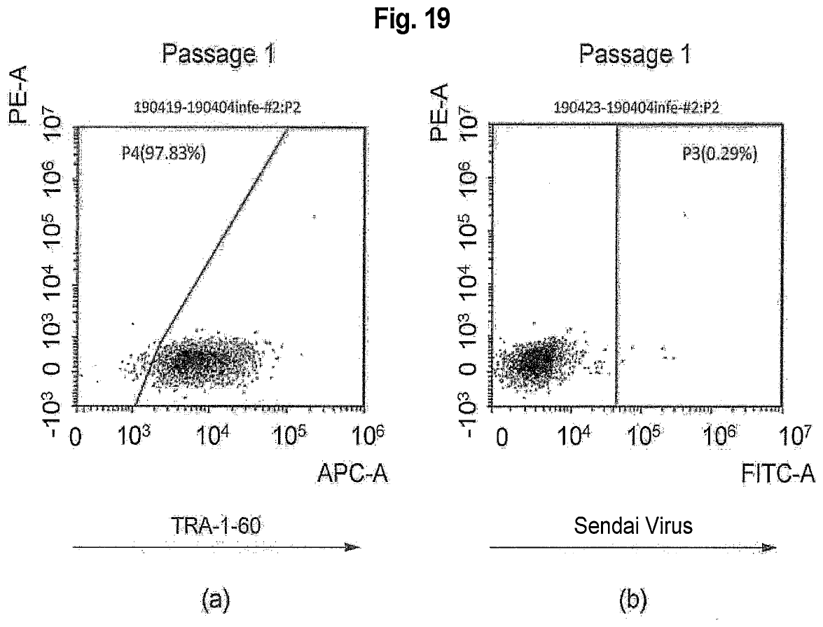

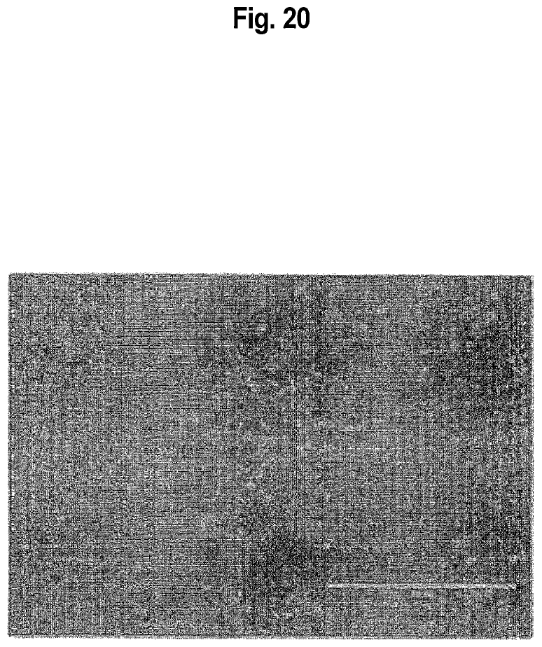

Using anti-Sendai virus antibodies, 15 days after infection, cells before passage were stained, the Sendai virus remaining in the cells was evaluated with a flow cytometer, and the results are shown in FIG. 17. In addition, FIG. 18 shows an image of the cells 15 days after infection. The cells that were passaged once were stained using anti-Sendai virus antibodies, the Sendai virus remaining in the cells was evaluated with a flow cytometer, and the results are shown in FIG. 19. As shown in FIG. 19, in the first passage, the Sendai virus in the cells almost disappeared. FIG. 20 shows an image of the cells in the first passage.

### Reference Example 5

SeV(PM) hKOS/TS12F, SeV18+hKLF4/TSΔF, and SeV(HNL)hC-Myc/TS15ΔF were added to mononuclear cells that were three-dimensionally cultured in a polymer-containing blood medium so that the MOI was 5, a dish for inducing pluripotent stem cells was accommodated in an incubator at 37° C. and the cells were cultured. Two days after infection, the polymer-containing blood medium was replaced with a polymer-containing iPS cell medium. Then, the medium was replaced once every two days using the polymer-containing iPS cell medium.

14 days after infection, stem-cell-like cell masses generated. On the 14th day after infection, almost all cells were TRA1-60 positive cells. Here, when some of the obtained TRA1-60 positive cells were seeded and two-dimensionally cultured in an incubator, iPS-cell-like colonies were formed. In addition, the cell masses were recovered using a mesh, a TrypLE select as a cell-releasing agent was added to the recovered cells, the cells were left at room temperature for 5 minutes, the cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Then, the well-dish was accommodated in an incubator at 37° C., and cells were two-dimensionally cultured. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other. Along the way, the temperature was raised to 38° C.

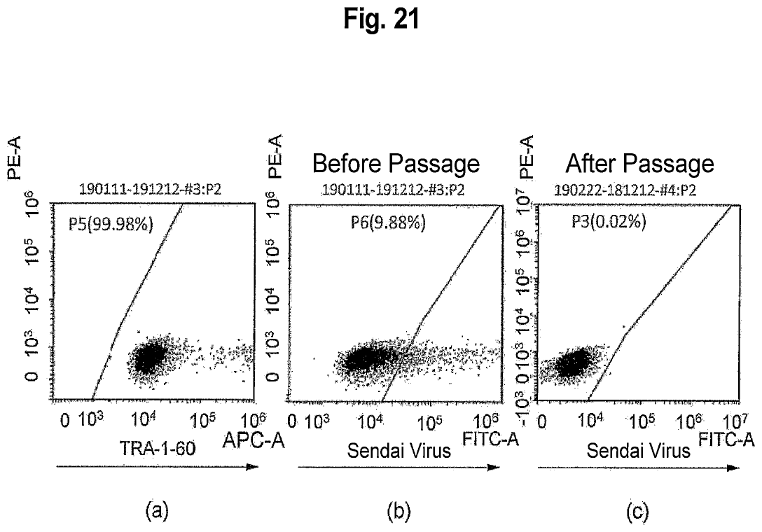

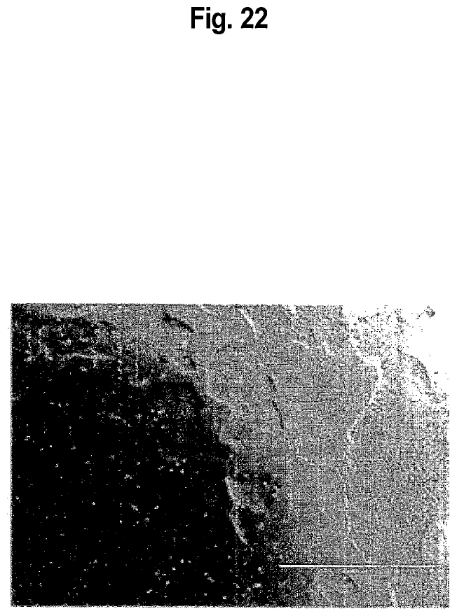

When the cells that were passaged twice were stained using anti-Sendai virus antibodies, and the Sendai virus remaining in the cells was evaluated with a flow cytometer, as shown in FIG. 21, the Sendai virus in the cells almost disappeared. FIG. 22 shows an image of the cells that were passaged twice.

### Reference Example 6

SeV(PM)hKOS/TS12ΔF and SeV(HNL)hC-Myc/TS15ΔF were added to two-dimensionally cultured fibroblasts so that the MOI was 5, a dish for inducing pluripotent stem cells was accommodated in an incubator at 34° C., and the cells were cultured. Two days after infection, the blood medium was replaced with an iPS cell medium. Then, the medium was replaced once every two days using the iPS cell medium.

8 days after infection, stem-cell-like cell masses generated. On the 14th day after infection, almost all cells become TRA1-60 positive cells, and showed iPS-cell-like morphology. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Then, the well-dish was accommodated in an incubator at 37° C., and cells were two-dimensionally cultured. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other.

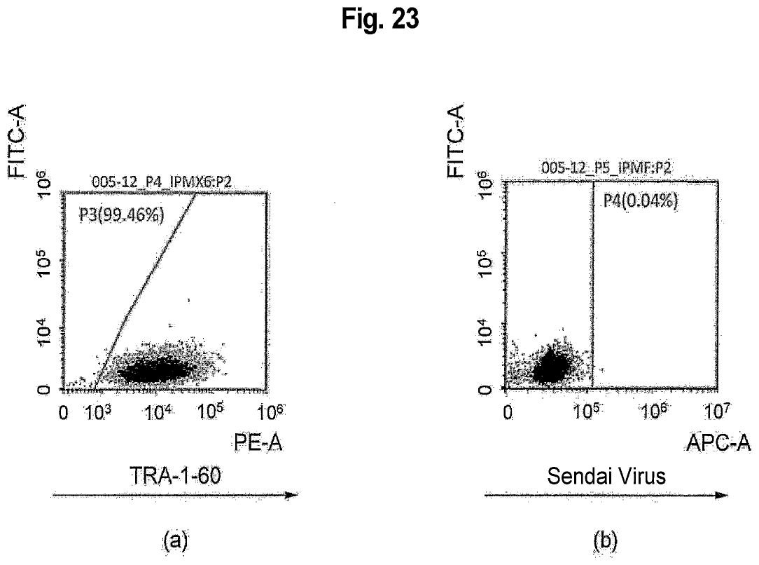

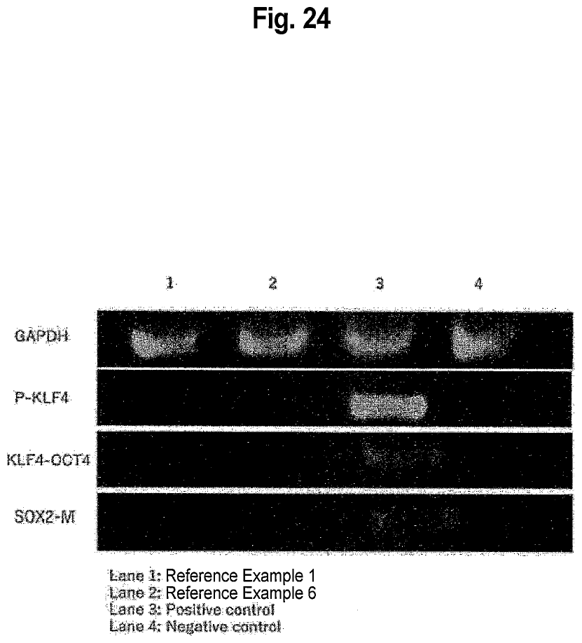

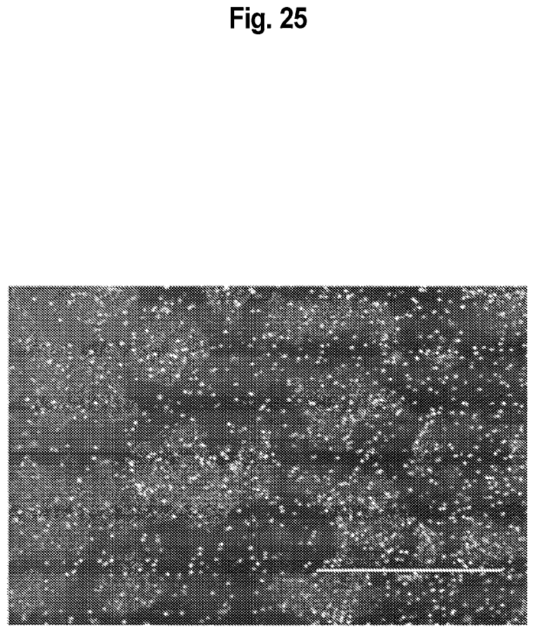

As shown in FIG. 23, when the cells were stained using anti-Sendai virus antibodies and the Sendai virus remaining in the cells was evaluated with a flow cytometer, the Sendai virus in the cells disappeared after the first passage. As shown in FIG. 24, the Sendai virus remaining in the cells was not detected by PCR. FIG. 25 shows an image of the obtained TRA1-60 positive cells.

### Example 7 and Comparative Example 7

A DMEM containing 10% FBS was prepared as a medium for fibroblasts. Fibroblasts were suspended in a medium for adult human-derived fibroblasts to obtain a fibroblast suspension.

A solution in which 1.5 mL of PBS and 4.8 μL of silkworm-derived laminin (iMatrix-511 silk, nippi) were mixed was added to one well of a 6-well dish, and the dish was left in an incubator at 37° C. for 1 hour. Next, a solution in which PBS and laminin were mixed was removed from the well using an aspirator, and 1.5 mL of the fibroblast suspension was added to one well. The number of fibroblasts in one well was 0.5×10⁵ to 2.0×10⁵. Then, the fibroblasts were cultured in an incubator at 37° C. for 1 day.

Next, the medium was replaced with a stem cell induction medium. The amount of the medium replaced was 1.5 mL.

A tube A and a tube B were prepared, and 0.1 μL and 100 μL of a mixture containing OCT4 RNA, SOX2 RNA, KLF4 RNA, and C-MYC RNA (100 ng/μL) was added to 125 μL of PBS in the tube A. These RNAs were concentrated and purified through HPLC. 0.1 μL to 100 μL of a lipofection reagent was added to 125 μL of PBS in the tube B. Next, the solution in the tube A and the solution in the tube B were mixed, the mixed solution was left at room temperature for 10 minutes, and a total amount of the mixed solution was added to the medium in one well. Then, the dish was left in an incubator at 37° C. for 1 day, and the cells were transfected with RNA. Then, RNA transfection was repeated 11 times according to the same procedures.

The day after the 11th RNA transfection, the concentration of the cell-containing solution was adjusted, and the cells were seeded in a laminin-coated well plate for the first passage so that the concentration was 0.25×10⁴ cells/cm² or less. In Example 7, all cells separated from the well plate were recovered during the first passage, and at least some of the recovered and mixed cells were seeded in the next well plate without distinguishing. On the other hand, in Comparative Example 7, colonies were picked and cloned during the first passage. Here, in both Example 7 and Comparative Example 7, cells were seeded so that 11 or more cells did not come into contact with each other during passage. Next, the well-dish was accommodated in an incubator at 37° C., and cells were two-dimensionally cultured. Then, the cells were passaged only once when the cells become 60% to 80% confluency. From the second passage onward, the cells were seeded in the well plate so that the concentration was 0.25×10⁴ cells/cm² or less. In this case, 11 or more cells did not come into contact with each other.

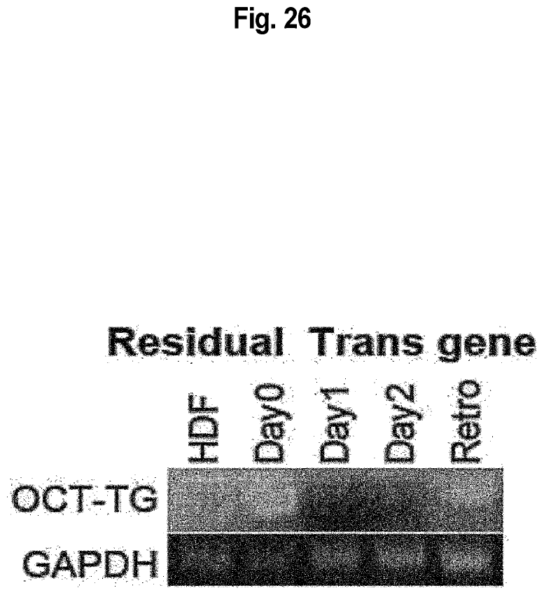

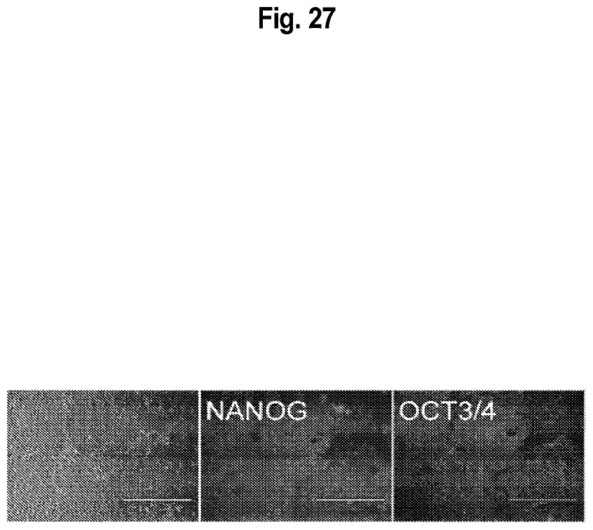

As shown in FIG. 26, on the first day after the first passage, the reprogramming factor almost disappeared from the cells, and on the second day after the first passage, the reprogramming factor completely disappeared from the cells. On the 10th day from infection, almost all cells became TRA1-60 positive cells, and showed iPS-cell-like morphology. FIG. 27 shows an immunostaining image of the TRA1-60 positive cells.

As in Example 1, the stem cells derived by the method according to Example 7 had a small variation in the clonal efficiency. As in Example 2, the stem cells derived by the method according to Example 7 had a stable proliferation rate and colony forming ability. As in Example 3, the stem cells derived by the method according to Example 7 had a small variation in the number of clamps and the number of cells after three-dimensional culture. As in Example 4, the stem cells derived by the method according to Example 7 had a stable ability to induce differentiation into cardiomyocytes. As in Example 5, the stem cells derived by the method according to Example 7 had a stable ability to induce differentiation into neural progenitor cells. As in Example 6, the stem cells derived by the method according to Example 7 had a stable ability to differentiate into the endoderm, the mesoderm, and the ectoderm.

As in Comparative Example 1, the stem cells derived by the method according to Comparative Example 7 had a large variation in the clonal efficiency. As in Comparative Example 2, the stem cells derived by the method according to Comparative Example 7 had a large variation in the proliferation rate and the colony forming ability. As in Comparative Example 3, the stem cells derived by the method according to Comparative Example 7 had a large variation in the number of clamps and the number of cells after three-dimensional culture. As in Comparative Example 4, the stem cells derived by the method according to Comparative Example 7 had an unstable ability to induce differentiation into cardiomyocytes. As in Comparative Example 5, the stem cells derived by the method according to Comparative Example 7 had an unstable ability to induce differentiation into neural progenitor cells. As in Comparative Example 6, the stem cells derived by the method according to Comparative Example 7 had an unstable ability to differentiate into the endoderm, the mesoderm, and the ectoderm.

### Example 8

As in Example 1, a reprogramming factor was introduced into mononuclear cells. On the 14th day after infection, almost all cells become TRA1-60 positive cells, and showed iPS-cell-like morphology. Then, in the stage in which cells were never passaged, iPS-cell-like cells were infected with the Sendai virus that can express Ngn2-Puro RNA so that the MOI was 20. On the second day after infection with the Sendai virus, the medium in the well was replaced with a nerve induction medium (N3 medium) containing puromycin at a concentration of 2 μg/mL, and uninfected cells were killed. The N3 medium was prepared by adding 10 mL of B27, 5 mL of N2, and 1.6 mL of insulin at a concentration of 6.25 mg/mL to 500 mL of DMEMF12.

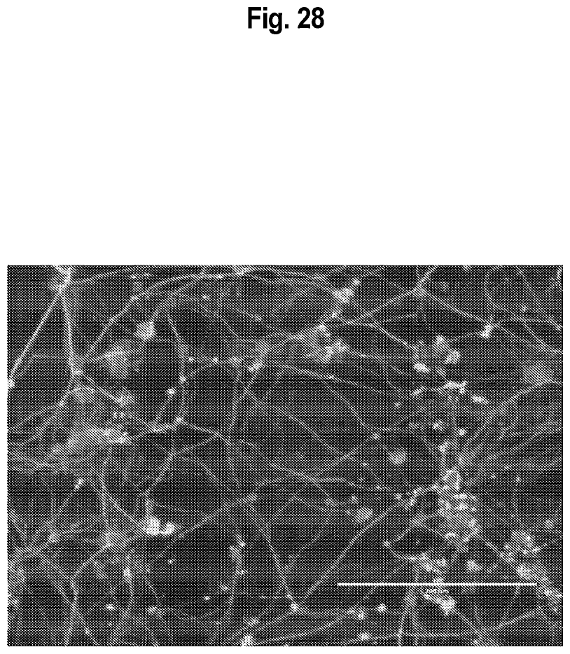

FIG. 28 shows a microscope image of the cells 14 days after infection with the Sendai virus. As shown in FIG. 28, it was morphologically confirmed that the cells were induced to nervous system cells after infection with the Sendai virus.

### Example 9

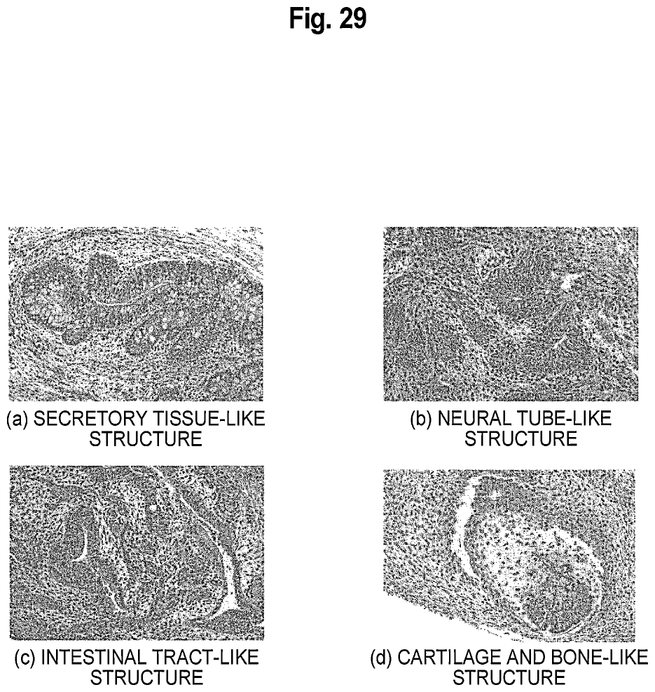

As in Example 1, a reprogramming factor was introduced into mononuclear cells. On the 14th day after infection, almost all cells become TRA1-60 positive cells, and showed iPS-cell-like morphology. Then, in the stage in which cells were never passaged, iPS-cell-like cells were transplanted into immunodeficient mouse testis. After a few weeks, teratomas were removed from a mouse, and a tissue section was prepared from the removed teratomas, and stained with hematoxylin and eosin (HE) and observed under a microscope. The results are shown in FIG. 29. A secretory tissue-like structure, a neural tube-like structure, an intestinal tract-like structure, a cartilage, and a bone-like structure were observed in the tissue section.

### Example 10

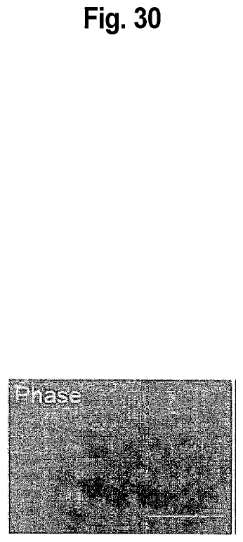

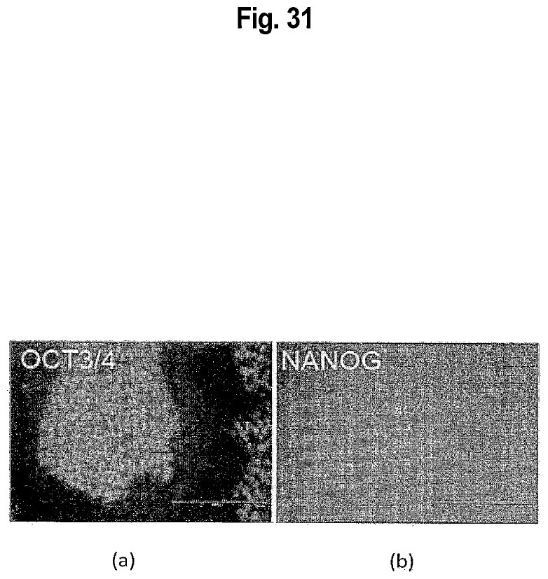

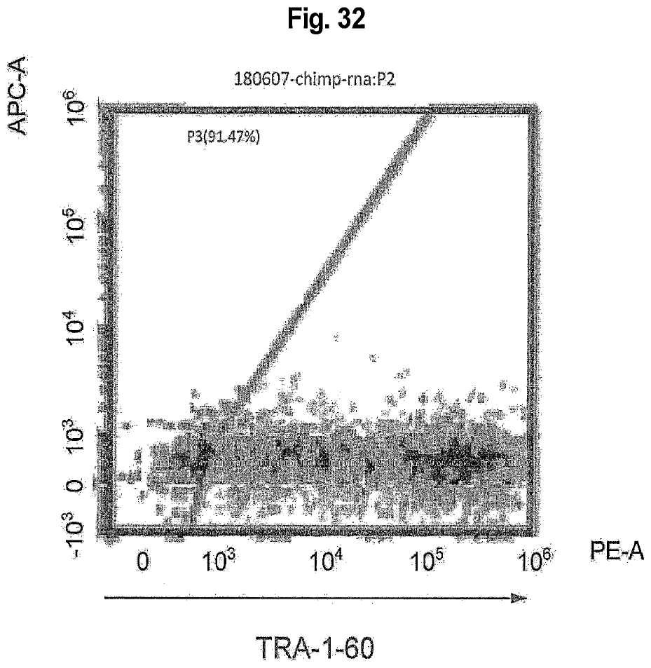

RNA was introduced into chimpanzee-derived fibroblasts in the same method as in Example 1 except that chimpanzee-derived fibroblasts were used. As a result, as shown in FIG. 30, formation of iPS-cell-like colonies was observed. When the maintenance-cultured cells were immunostained with antibodies for Oct3/4, as shown in FIG. 31(a), the cells showed Oct3/4 positive. In addition, when the maintenance-cultured cells were immunostained with antibodies for Nanog, as shown in FIG. 31(b), the cells showed Nanog positive. In addition, as shown in FIG. 32, it was confirmed that the induced cells were TRA-1-60 positive.

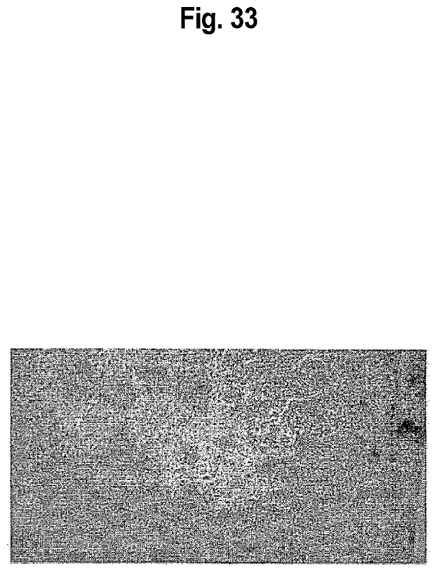

The derived chimpanzee-derived stem cells were induced to differentiate into cardiomyocytes. FIG. 33 shows an image of the obtained cardiomyocytes.

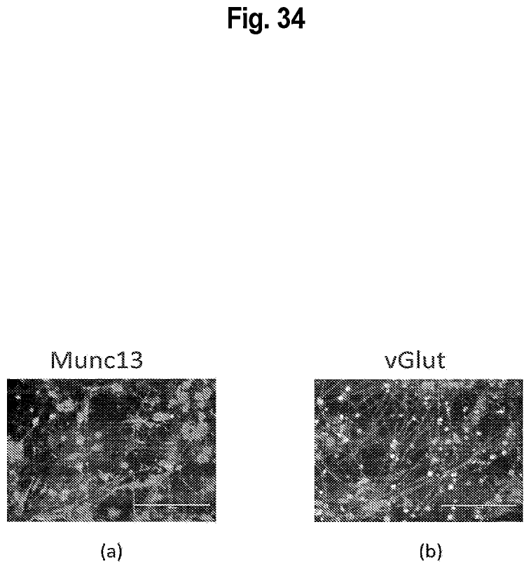

In addition, the derived chimpanzee-derived stem cells were induced to differentiate into nerve cells. FIG. 34 shows images of the obtained cardiomyocytes. It was confirmed that the nerve cells were Munch13 positive and vGlut positive.

### Example 11

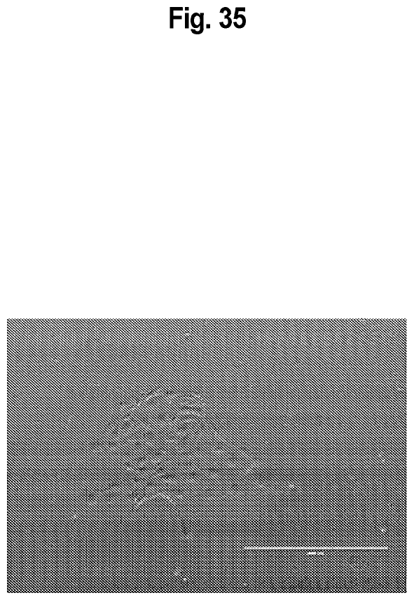

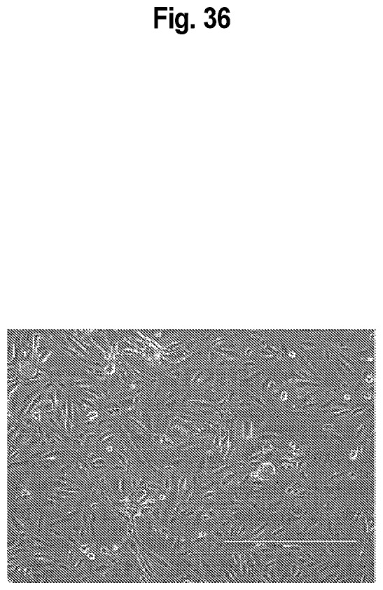

300 mL of urine was collected from a healthy subject, 6 urine samples were dispensed into a 50 mL Falcon tube, and the tube was centrifuged at 400G for 5 minutes. After centrifugation, the supernatant was removed from the tube, 30 mL of PBS was put into the tube, and the tube was centrifuged at 400G for 5 minutes. After centrifugation, the supernatant was removed from the tube, 30 mL of a primary medium was put into the tube, and the tube was centrifuged at 400G for 5 minutes. A primary medium was prepared by adding fetal bovine serum (Gibco, 10437028, final concentration 10%), SingleQuots Kit CC-4127 REGM (Lonza, 1/1000 amount), and Antibiotic-Antimycotic (Gibco, 15240062, 1/100 amount) to DMEM/Ham's F12 (Gibco, 11320-033). After centrifugation, the supernatant was removed from the tube, cells were suspended in 1 mL of the primary medium, the cells were seeded in one well of a gelatin-coated 24-well plate, and the cells were incubated in an incubator at 37° C. For 2 days after the cells were seeded, the primary medium was added to a 300 μL well, and from the 3^{rd} day onward, the medium was replaced using a medium for epithelial cells. The medium for epithelial cells was prepared by adding SingleQuots Kit CC-4127 REGM (Lonza) to a renal epithelial cell basal medium (Lonza) FIG. 35 shows a microscope image of the cells after expansion-cultured for 6 days. The cells were subjected to the first passage on the 7th day after seeding, the cells were additionally expansion-cultured, and the cells were subjected to the second passage on the 7th day after the first passage. FIG. 36 shows a microscope image of the cells on the 6th day after the second passage.

### Example 12

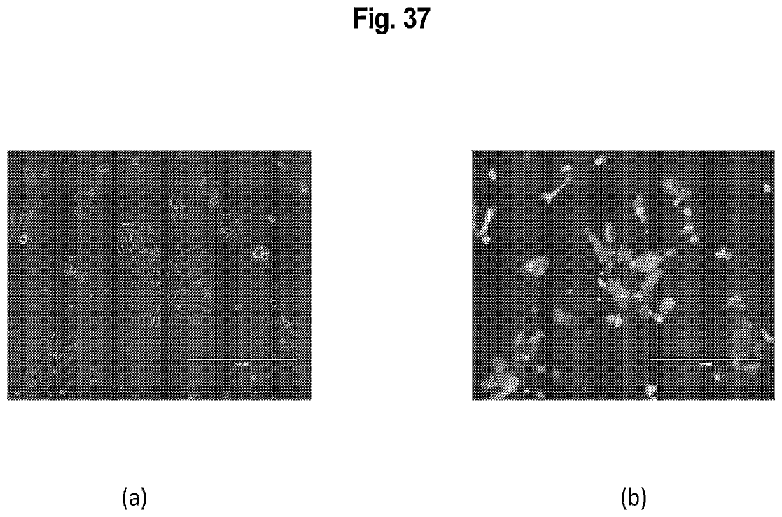

A dish coated with laminin 511 was used as a dish for inducing pluripotent stem cells. 1×10⁴ to 1×10⁵ urine-derived cells prepared in Example 11 were seeded in the dish for inducing pluripotent stem cells and incubated at 37° C. For the medium, a medium for epithelial cells was used. The next day, a mixture containing a transfection reagent and RNA that encodes a green fluorescent protein (GFP) was added to a medium, the medium was replaced with the above medium, and incubated at 37° C. FIG. 37 shows microscope images of the cells on the next day. Expression of GFP was observed, which indicates that transfection into urine-derived cells was performed.

### Example 13

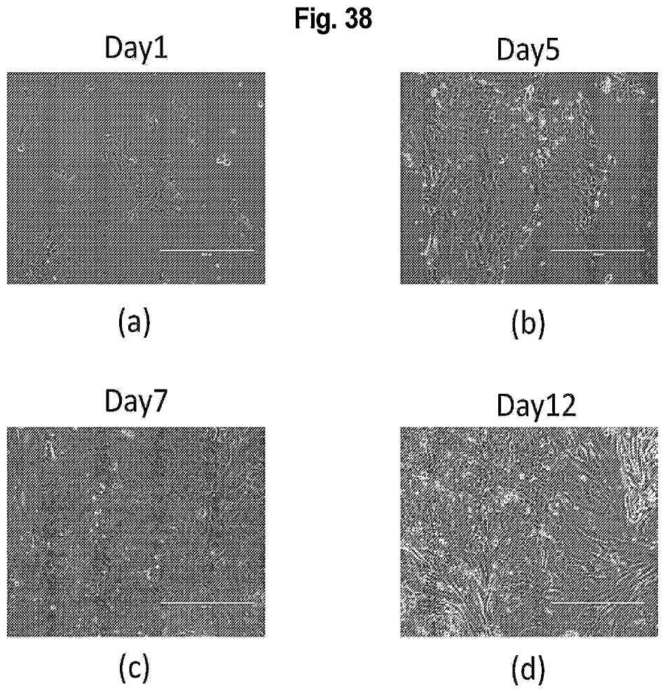

A dish coated with laminin 511 was used as a dish for inducing pluripotent stem cells. 1×10⁴ to 1×10⁵ urine-derived cells prepared in Example 11 were seeded in the dish for inducing pluripotent stem cells and incubated at 37° C. For the medium, a medium for epithelial cells was used. The next day, a tube A and a tube B were prepared, and 0.1 μL to 10² μL of a mixture containing M₃O RNA, SOX2 RNA, KLF4 RNA, C-MYC RNA, and LIN28 RNA (100 ng/μL) was added to 125 μL of PBS in the tube A. These RNAs were modified with pseudouridine (ψ). In addition, these RNAs were substantially concentrated and purified into single-stranded RNA through HPLC. It was confirmed that the ratio (A₂₆₀/A₂₈₀) of the absorbances of RNA at 260 nm and 280 nm was 1.71 to 2.1, and proteins were not substantially mixed. In addition, when dot blot analysis was performed using anti-double-stranded RNA antibody J2, 90% or more of double-stranded RNA was removed. 0.1 μL to 100 μL of a lipofection reagent was added to 125 μL of PBS in the tube B. Next, the solution in the tube A and the solution in the tube B were mixed, the mixed solution was left at room temperature for 10 minutes, a total amount of the mixed solution was added to a transfection medium without using B18R or the like, the medium was replaced using the transfection medium, and incubated at 37° C. Transfection was performed once daily for 10 days. Observation was performed on days 1, 5, 7, and 14 after cell seeding. As shown in FIG. 38, it was observed that cell morphology changed like ES cells as the day progressed.

### Example 14

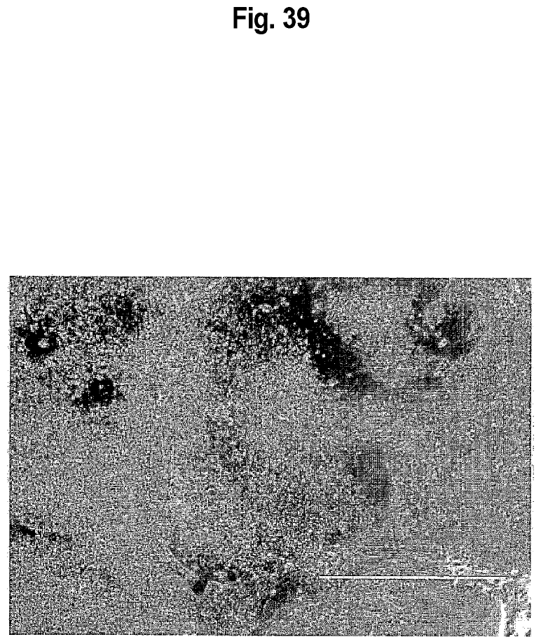

As in Example 13, urine-derived cells were transfected for 10 days. The cells were cultured in a medium for stem cells (StemFit, Ajinomoto) from the 11th day after the cells were seeded, all cells were separated from the dish on the 14th day, and some of the recovered and mixed cells were seeded and passaged in the medium. During passage, without picking up colonies, all cells on the dish were recovered, and 1×10² to 1×10⁵ cells were seeded on the dish. FIG. 39 shows a microscope image of the cells 6 days after passage. ES-cell-like cells were confirmed.

### Example 15

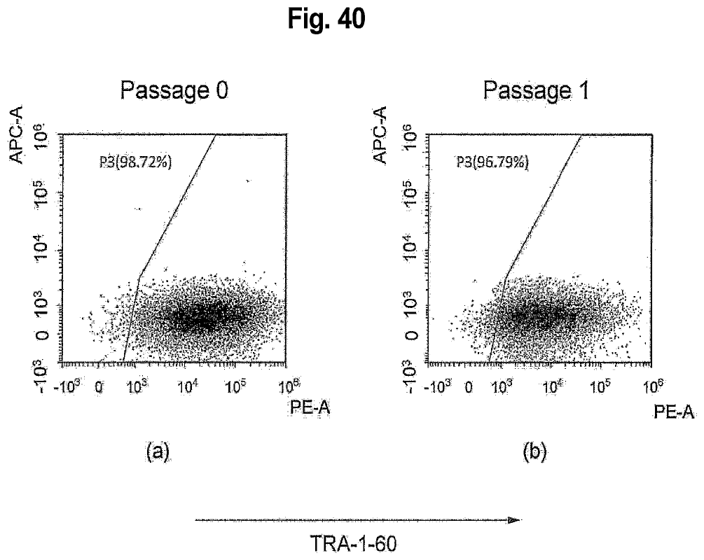

As in Example 13, urine-derived cells were transfected for 10 days. When the cells were cultured in a medium for stem cells (StemFit, Ajinomoto) from the 11th day after the cells were seeded, the cells were separated from the dish on the 14th day, and some of the cells were analyzed using a flow cytometry, as shown in FIG. 40(a), TRA-1-60 positive was confirmed. In addition, when the cells separated from the dish on the 14th day were passaged, and analyzed using a flow cytometry 7 days later, as shown in FIG. 40(b), TRA-1-60 positive was confirmed.

### Example 16

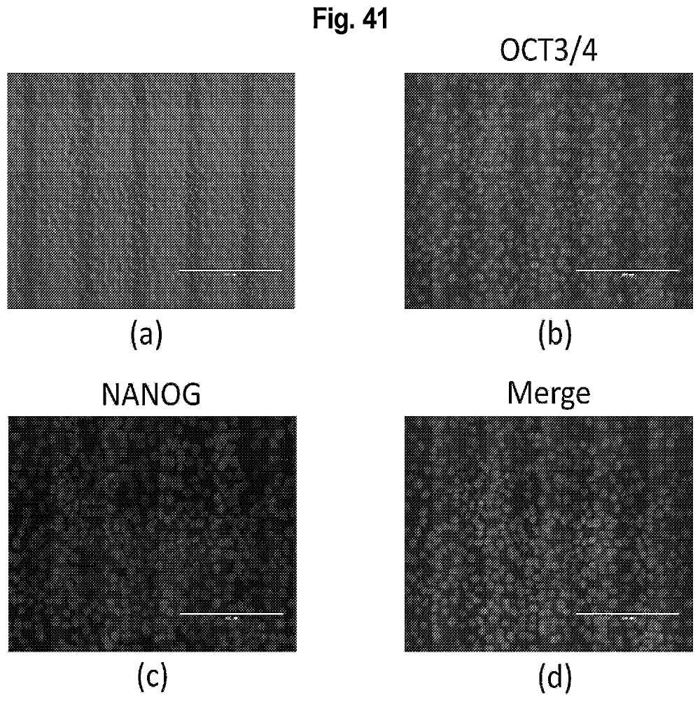

As in Example 13, urine-derived cells were transfected for 10 days. The cells were cultured in a medium for stem cells (StemFit, Ajinomoto) from the 11th day after the cells were seeded, all cells were separated from the dish on the 14th day, and some of the separated and mixed cells were seeded and passaged. StemFit (registered trademark) was used as the medium after passage. 7 days after passage, cells were immobilized, and the cells were stained using anti-OCT3/4 antibodies and anti-NANOG antibodies. In addition, nuclei chemical staining using Hoechst (registered trademark) was performed. As a result, as shown in FIG. 41, expression of OCT3/4 and NANOG, which are specific markers for pluripotent stem cells, in cell nuclei was confirmed. Therefore, it was shown that the pluripotent stem cells could be induced from urine-derived cells using RNA. Here, FIG. 41(d) is an image obtained by synthesizing an image of cells stained using anti-OCT3/4 antibodies, an image of cells stained using anti-NANOG antibodies, and an image of cells stained using Hoechst (registered trademark).

### Example 17

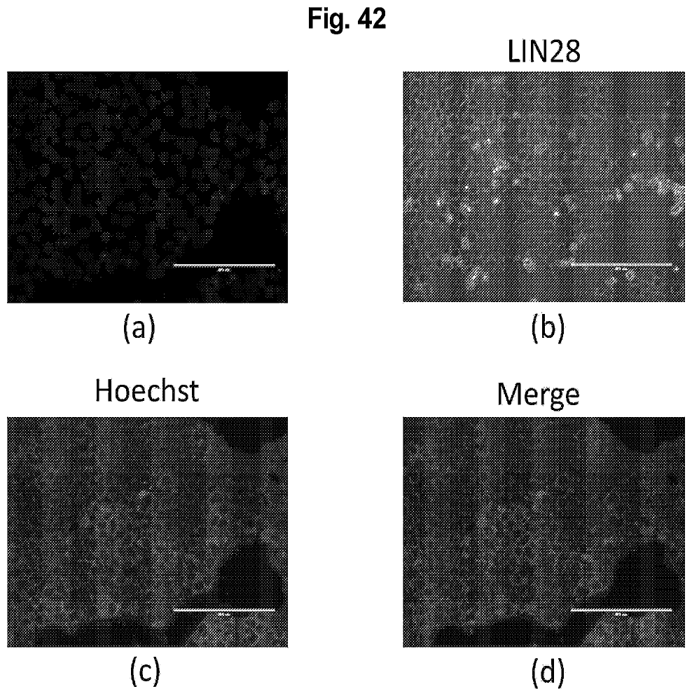

As in Example 13, urine-derived cells were transfected for 10 days. The cells were cultured in a medium for stem cells (StemFit, Ajinomoto) from the 11th day after the cells were seeded, all cells were separated from the dish using a TrypLE select on the 14th day, and some of the separated and mixed cells were seeded and passaged in a medium. StemFit (registered trademark) was used as the medium after passage. 7 days after passage, cells were immobilized, and the cells were stained using anti-LIN28 antibodies. In addition, nuclei chemical staining using Hoechst (registered trademark) was performed. As a result, as shown in FIG. 42, expression of LIN28, which is a specific marker for pluripotent stem cells, in cell nuclei was confirmed. Therefore, it was shown that the pluripotent stem cells could be induced from urine-derived cells using RNA. Here, FIG. 42(d) is an image obtained by synthesizing an image of cells stained using anti-LIN28 antibodies and an image of cells stained using Hoechst (registered trademark).

1. A method for culturing factor-introduced cells without cloning cells.

2.-6. (canceled)

7. The method according to claim 1,
wherein, in the seeding, the factor-introduced cells are mixed.

8. The method according to claim 1,
wherein, in the seeding, clones of the factor-introduced cells are mixed.

9. The method according to claim 1,
wherein, in the seeding, different clones of the factor-introduced cells are mixed.

10. The method according to claim 1, which does not include isolating a plurality of colonies, which are formed by the factor-introduced cells, before the seeding.

11. The method according to claim 1,
wherein, in the seeding, a plurality of colonies formed by the factor-introduced cells are mixed with each other.

12. The method according to claim 1, which does not include cloning a single colony, which is formed by the factor-introduced cells, before the seeding.

13. The method according to claim 1,
wherein cells which are attached to an incubator and which are the factor-introduced cells, are recovered and at least part of the recovered cells are seeded in a medium.

14. The method according to claim 1,
wherein the factor-introduced cells are seeded without distinguishing the cells according to a gene expression state thereof.

15. The method according to claim 1,
wherein the factor-introduced cells are seeded without distinguishing the cells according to a degree of reprogramming.

16. The method according to claim 1,
wherein the seeded cells are induced to pluripotent stem cells.

17. The method according to claim 16, further comprising inducing the pluripotent stem cells to somatic cells.

18. The method according to claim 1, further comprising freezing the factor-introduced cells.

19. The method according to claim 1, further comprising differentiating the factor-introduced cells into at least one selected from among the endoderm, the mesoderm, and the ectoderm.

20. The method according to claim 1, further comprising forming at least one selected from among embryoid bodies, organoids, and spheres from the factor-introduced cells.

21. The method according to claim 1, further comprising inducing the factor-introduced cells to somatic cells different from pluripotent stem cells.

22. The method according to claim 21, further comprising cloning inducement-treated cells after a process of inducement to somatic cells.

23. The method according to claim 1, further comprising performing a gene editing process on the factor-introduced cells.

24. The method according to claim 1,
wherein the factor-introduced cells are derived from blood cells, fibroblasts, or cells contained in urine.

25. The method according to claim 1,
wherein the factor-introduced cells are derived from a plurality of humans or a plurality of non-human animals.

26. The method according to claim 1, comprising culturing the factor-introduced cells in a closed incubator.

27. The method according to claim 1, comprising:
culturing factor-introduced cells; and
inducing the factor-introduced cells to somatic cells different from pluripotent stem cells without passaging.

28. The method according to claim 27, further comprising freezing the factor-introduced cells.

29. The method according to claim 27, further comprising inducing the factor-introduced cells to at least one selected from among the endoderm, the mesoderm, and the ectoderm.

30. The method according to claim 27, comprising culturing the factor-introduced cells in a closed incubator.
